(19) United States
(12) Patent Application Publication
(10) Pub. No.: US 2022/0175851 A1
(43) Pub. Date: Jun. 9, 2022

(54) COMPOSITIONS, METHODS FOR REGULATING UTERINE, PLACENTAL GROWTH

(71) Applicant: The Regents of the University California, Oakland, CA (US)

(72) Inventors: Elaine Y. Hsiao, Los Angeles, CA (US); Geoffrey N. Pronovost, Los Angeles, CA (US)

(21) Appl. No.: 17/437,643

(22) PCT Filed: Mar. 13, 2020
(86) PCT No.: PCT/US20/22637

§ 371 Date: Sep. 9, 2021

Related U.S. Application Data
(60) Provisional application No. 62/817,629, filed on Mar. 13, 2019.

(51) Int. Cl. A61K 35/74; A61P 15/00; A61P 3/02; A61K 31/575; A61K 31/353; A61K 38/18

(52) U.S. Cl. CPC A61K 35/74; A61P 15/00; A61P 3/02; A61K 38/18; A61K 31/353; A61K 38/1875; A61K 31/575

(57) ABSTRACT

Disclosed herein are methods for promoting fetal growth, placental development, and angiogenesis, comprising administering to a female subject or to a maternal subject gestating an unborn baby (e.g., fetus) a composition of the present disclosure. Also disclosed are methods of inhibiting development of a disease or disorder, such as preeclampsia or fetal growth reduction (FGR), in a subject, comprising administering to a female subject or to a maternal subject gestating an unborn baby (e.g., fetus) a composition of the present disclosure.

### RELATED APPLICATION

This application claims a right of priority to and the benefit of U.S. Provisional Application No. 62/817,629, filed on Mar. 13, 2019, which is hereby incorporated by reference in its entirety.

### STATEMENT REGARDING FEDERALLY SPONSORED RESEARCH

This invention was made with government support under Grant Number A1007323, awarded by the National Institutes of Health. The government has certain rights in the invention.

### BACKGROUND

Pregnancy consists of several complex interactions between maternal and fetal cells, many of which occur at the placenta, or literal maternal-fetal interface. The placenta is a dynamic organ, comprised of maternal vasculature juxtaposed with dense fetal vasculature to mediate transport of maternal factors to the rapidly developing fetus. The placenta is also a critical immune interface; the maternal compartment of the placenta is densely packed with immune cells, dominated by specialized uterine natural killer cells (uNKs). Many gestational challenges, such as maternal malnutrition, stress, and immune activation, disrupt placental immunity and vascularization, and predispose mother and fetus to disorders such as preeclampsia and fetal growth restriction (FGR). However, the molecular mechanisms that control placental immune function and vascular development are not fully understood. Moreover, exactly how environmental exposures during gestation yield lasting impact on offspring health remains unclear.

Multiple studies have demonstrated the microbiome's capacity to regulate the vasculature, including angiogenesis and barrier function. The microbiome has also been shown to regulate host growth in adult animals through insulin-like growth factor-1 (IGF1), and bacterial taxa enriched in severely malnourished humans dysregulated host metabolism following transplantation in gnotobiotic mice.

Thus, methods of modifying the maternal microbiome, e.g., to compensate for a depleted maternal microbiome, prenatally (i.e., during gestation) are needed.

### SUMMARY

Disclosed herein are methods of promoting healthy growth in an unborn baby (e.g., fetus).

In certain embodiments, the present invention provides methods comprising administering to a maternal subject gestating an unborn baby (e.g., fetus) a composition comprising: a compound selected from primary bile acids, secondary bile acids, daidzein, genistein, osteopontin, osteoglycin, pleiotrophin, tyrosine, histidine, peptides, benzoate, tryptophan, 2-aminophenol sulfate, catechol sulfate, 4-hydroxyphenylacetate sulfate, N,N,N-trimethyl-5-aminovalerate, phenol sulfate, equol sulfate, hippurate, p-cresol sulfate, imidazolepropionate, indolepropionate, equol glucuronide, valerylglycine, phenol glucuronide, salicylate, S-carboxymethyl-L-cysteine, and trimethylamine N-oxide, and metabolites and derivatives thereof, or a combination thereof; and/or one or more bacterial species found in a maternal microbiome, selected from phylum Firmicutes, phylum Bacteroidetes, phylum Proteobacteria, or a combination thereof.

In certain embodiments, the present invention provides methods comprising administering to a female subject a composition comprising: a compound selected from primary bile acids, secondary bile acids, daidzein, genistein, osteopontin, osteoglycin, pleiotrophin, tyrosine, histidine, peptides, benzoate, tryptophan, 2-aminophenol sulfate, catechol sulfate, 4-hydroxyphenylacetate sulfate, N,N,N-trimethyl-5-aminovalerate, phenol sulfate, equol sulfate, hippurate, p-cresol sulfate, imidazolepropionate, indolepropionate, equol glucuronide, valerylglycine, phenol glucuronide, salicylate, S-carboxymethyl-L-cysteine, and trimethylamine N-oxide, and metabolites and derivatives thereof, or a combination thereof and/or one or more bacterial species found in a maternal microbiome, selected from phylum Firmicutes, phylum Bacteroidetes, phylum Proteobacteria, or a combination thereof wherein the female subject is a fertile, ovulating female subject or a female subject seeking to implant an embryo.

Also disclosed herein are methods of promoting placental development in a maternal subject gestating an unborn baby (e.g., fetus).

In certain embodiments, the present invention provides methods comprising administering to a maternal subject gestating an unborn baby (e.g., fetus) a composition comprising: a compound selected from primary bile acids, secondary bile acids, daidzein glucuronide, genistein glucuronide, osteopontin, osteoglycin, and pleiotrophin, tyrosine, histidine, peptides, benzoate, tryptophan, 2-aminophenol sulfate, catechol sulfate, 4-hydroxyphenylacetate sulfate, N,N,N-trimethyl-5-aminovalerate, phenol sulfate, equol sulfate, hippurate, p-cresol sulfate, imidazolepropionate, indolepropionate, equol glucuronide, valerylglycine, phenol glucuronide, salicylate, S-carboxymethyl-L-cysteine, and trimethylamine N-oxide, and metabolites and derivatives thereof, or a combination thereof; and/or one or more bacterial species found in a maternal microbiome, selected from phylum Firmicutes, phylum Bacteroidetes, phylum Proteobacteria, or a combination thereof.

In certain embodiments, the present invention provides methods comprising administering to a female subject a composition comprising: a compound selected from primary bile acids, secondary bile acids, daidzein, genistein, osteopontin, osteoglycin, pleiotrophin, tyrosine, histidine, peptides, benzoate, tryptophan, 2-aminophenol sulfate, catechol sulfate, 4-hydroxyphenylacetate sulfate, N,N,N-trimethyl-5-aminovalerate, phenol sulfate, equol sulfate, hippurate, p-cresol sulfate, imidazolepropionate, indolepropionate, equol glucuronide, valerylglycine, phenol glucuronide, salicylate, S-carboxymethyl-L-cysteine, and trimethylamine N-oxide, and metabolites and derivatives thereof, or a combination thereof; and/or one or more bacterial species found in a maternal microbiome, selected from phylum Firmicutes, phylum Bacteroidetes, phylum Proteobacteria, or a combination thereof; wherein the female subject is a fertile, ovulating female subject or a female subject seeking to implant an embryo.

Also disclosed herein are methods of promoting angiogenesis in a fetal subject.

In certain embodiments, the present invention provides methods comprising administering to a maternal subject gestating the unborn baby (e.g., fetus) a composition comprising: a compound selected from primary bile acids, secondary bile acids, daidzein, genistein, osteopontin, osteoglycin, pleiotrophin, tyrosine, histidine, peptides, benzoate, tryptophan, 2-aminophenol sulfate, catechol sulfate, 4-hydroxyphenylacetate sulfate, N,N,N-trimethyl-5-aminovalerate, phenol sulfate, equol sulfate, hippurate, p-cresol sulfate, imidazolepropionate, indolepropionate, equol glucuronide, valerylglycine, phenol glucuronide, salicylate, S-carboxymethyl-L-cysteine, and trimethylamine N-oxide, and metabolites and derivatives thereof, or a combination thereof; and/or one or more bacterial species found in a maternal microbiome, selected from phylum Firmicutes, phylum Bacteroidetes, phylum Proteobacteria, or a combination thereof.

In certain embodiments, the present invention provides methods comprising administering to a female subject a composition comprising: a compound selected from primary bile acids, secondary bile acids, daidzein, genistein, osteopontin, osteoglycin, pleiotrophin, tyrosine, histidine, peptides, benzoate, tryptophan, 2-aminophenol sulfate, catechol sulfate, 4-hydroxyphenylacetate sulfate, N,N,N-trimethyl-5-aminovalerate, phenol sulfate, equol sulfate, hippurate, p-cresol sulfate, imidazolepropionate, indolepropionate, equol glucuronide, valerylglycine, phenol glucuronide, salicylate, S-carboxymethyl-L-cysteine, and trimethylamine N-oxide, and metabolites and derivatives thereof, or a combination thereof and/or one or more bacterial species found in a maternal microbiome, selected from phylum Firmicutes, phylum Bacteroidetes, phylum Proteobacteria, or a combination thereof wherein the female subject is a fertile, ovulating female subject or a female subject seeking to implant an embryo.

Also disclosed herein are methods of inhibiting development of preeclampsia or Fetal Growth Restriction (FGR) in a maternal subject gestating an unborn baby (e.g., fetus).

In certain embodiments, the present invention provides methods comprising administering to a maternal subject gestating an unborn baby (e.g., fetus) a composition comprising: a compound selected from primary bile acids, secondary bile acids, daidzein, genistein, osteopontin, osteoglycin, pleiotrophin, tyrosine, histidine, peptides, benzoate, tryptophan, 2-aminophenol sulfate, catechol sulfate, 4-hydroxyphenylacetate sulfate, N,N,N-trimethyl-5-aminovalerate, phenol sulfate, equol sulfate, hippurate, p-cresol sulfate, imidazolepropionate, indolepropionate, equol glucuronide, valerylglycine, phenol glucuronide, salicylate, S-carboxymethyl-L-cysteine, and trimethylamine N-oxide, and metabolites and derivatives thereof, or a combination thereof; and/or one or more bacterial species found in a maternal microbiome, selected from phylum Firmicutes, phylum Bacteroidetes, phylum Proteobacteria, or a combination thereof.

In certain embodiments, the present invention provides methods comprising administering to a female subject a composition comprising: a compound selected from primary bile acids, secondary bile acids, daidzein, genistein, osteopontin, osteoglycin, pleiotrophin, tyrosine, histidine, peptides, benzoate, tryptophan, 2-aminophenol sulfate, catechol sulfate, 4-hydroxyphenylacetate sulfate, N,N,N-trimethyl-5-aminovalerate, phenol sulfate, equol sulfate, hippurate, p-cresol sulfate, imidazolepropionate, indolepropionate, equol glucuronide, valerylglycine, phenol glucuronide, salicylate, S-carboxymethyl-L-cysteine, and trimethylamine N-oxide, and metabolites and derivatives thereof, or a combination thereof and/or one or more bacterial species found in a maternal microbiome, selected from phylum Firmicutes, phylum Bacteroidetes, phylum Proteobacteria, or a combination thereof wherein the female subject is a fertile, ovulating female subject or a female subject seeking to implant an embryo.

In any of the embodiments, the pharmaceutical compositions/preparations can include any one or more of the following: imidazole propionate; N,N,N-trimethyl-5-aminovalerate; 4-hydroxyphenylacetate; phenol sulfate; indolepropionate; indoxyl glucuronide; N-methylproline; phenylacetylglycine; trimethylamine N-oxide; taurodeoxycholate; biotin; hippurate; 2-(4-hydroxyphenyl)propionate; cinnamoylglycine; equol glucuronide; equol sulfate; 2-aminophenol sulfate; 3-indoxyl sulfate; p-cresol sulfate.

Such pharmaceutical preparations may be for use in treating or preventing a condition or disease as described herein.

### BRIEF DESCRIPTION OF THE FIGURES

FIG. 1: The placenta regulates maternal-fetal exchange, facilitating transport of biochemical essential for fetal growth.

FIG. 2: Critical events during gestation.

FIG. 3: The maternal microbiota regulates placental and fetal development. (A) Average E14.5 placental weights from SPF (n=10), GF (n=8), ABX (n=10) and GF-CONV (n=7) litters. (B) Average E14.5 fetal weights from litters represented in (A). (C) Fetal to placental weight ratios from litters used in (A-B). (D) Correlation analysis of litters represented in (A-B). (E) Average P0 neonatal weights of SPF (n=6), GF (n=6), ABX (n=5) and GF-CONV (n=2). (F) Representative images hematoxyling and eosin staining of E14.5 SPF, GF and ABX placentae. (G) Enlarged inset images from hematoxyling and eosin staining of E14.5 fetal labyrinth displayed in (F). (H) Representative images of E14.5 SPF, GF, ABX and GF-CONV fetuses reconstructed using micro-computed tomography (μCT) and quantification of total fetal volume (I), fetal brain volume (K), fetal brain ventricle volume (K), brain to body volume ratios (L), and fetal surface area (M).

FIG. 4: The maternal microbiota regulates placental and fetal development. (A-E) Analysis of individual placentae and fetuses from litters described in FIG. 3. (F) Maternal weight gain assessed at E0.5, E7.5, E13.5 and E14.5 in SPF (n=9), GF (n=2), ABX (n=7) and GF-CONV (n=6) dams. (G) Representative images of gross morphological features of E14.5 SPF, GF and ABX fetuses (scale=1 cm).

FIG. 5: Reduced placental and fetal weight is not due to off-target effects of absorbable antibiotics. (A) Average placental weights from E14.5 SPF (same as FIG. 3A), ABX (n=9), non-absorbable antibiotic treated (Non-Abs; n=4) and absorbable-antibiotic treated (Abs; n=5) litters. (B) Average E14.5 fetal weights from litters represented in (A). (C) Average fetal to placental weight ratios from litters used in (A-B). (D) Correlation analysis of individual placentae and fetuses in litters represented in (A-B). (E-G) Analyses of individual placentae and fetuses from litter group analyses represented in (A-C). (H) Maternal weight gain assessed at E0.5, E7.5, E13.5 and E14.5 in ABX (n=9), Non-abs (n=4) and Abs (n=3) dams.

FIG. 6: Candidate bacterial taxa associated with reduced placental and fetal growth deficits revealed by 16S sequencing. (A) Taxonomic identities of bacterial 16S ribosomal DNA sequencing isolated from E14.5 SPF (n=3), non-absorbable antibiotic treated (Non-Abs; n=5), and absorbable antibiotic treated (Abs; n=5) fecal samples. (B) Alpha diversity rarefaction plots from samples described in (A). (C) Beta diversity determined by weighted-Unifrac analysis of samples described in (A).

FIG. 7: Critical events in uNK dynamics.

FIG. 8: Depletion of the maternal microbiota alters uterine natural killer (uNK) abundance in the E14.5 decidua. (A) Representative images of E14.5 deciduae from SPF, GF and ABX mice stained against Dolichos Biflorus Aggultinin-lectin (DBA) and DAPI. (B) Representative images of E14.5 deciduae from SPF, GF and ABX mice stained against DBA and Eomesodermin (Eomes). (C) Quantification of images represented in (A-B), demonstrating numbers of cells stained positive for DBA only and for DBA co-localized with Eomes. (D) Representative frequencies of DBA expression in E14.5 SPF, GF and ABX deciduae, gated on live CD45+, CD3-, CD122+ cells. (E) Quantification of DBA positive and negative frequencies as shown in (D) from SPF (n=5), GF (n=5) and ABX (n=6) deciduae. (F) Histological analysis of gd14.5 decidua stained against DAPI (blue) and DBA-lectin (green). (G) Histological analysis and quantification (H) of decidua stained against DBA-lectin (green) and Eomes (magenta) (SPF n=8, GF n=8, ABX n=9). As seen trhough these panels, the maternal microbiome regulates angiogenic uterine natural killer abundance in gd14.5 decidua.

FIG. 9: Maternal microbiota depletion does not alter uNK maturation phenotypes. (A) Frequency of CD45+ cells in E14.5 deciduae from SPF (n=5), GF (n=5) and ABX (n=8) mice, determined by flow cytometry. (B) Frequency of CD3− CD122+ cells in E14.5 deciduae from SPF (n=5), GF (n=5) and ABX (n=8) mice, determined by flow cytometry. (C) Expression of uNK markers CD69, KLRG1, CD11b and Ly49D from SPF (n=5), GF (n=5) and ABX (n=8) E14.5 deciduae.

FIG. 10: Critical events in placental vascular development.

FIG. 11: The maternal microbiota promotes placental vascular development. (A) Representative feto-placental arterial vascular reconstructions by micro-computed tomography of E14.5 SPF, GF, ABX and GF-CONV placentae. Quantification of E14.5 feto-placental arterial vasculature from SPF (n=9), GF (n=9), ABX (n=6) and GF-CONV (n=5) volume (B) and surface area (C). (D) Histological analysis of feto-placental microvasculature in E14.5 SPF, GF and ABX placentae stained against laminin, TER119 and DAPI. (E) Quantification of raw integrated density of laminin staining in the fetal labyrinth in SPF (n=6), GF (n=7) and ABX (n=6) placentae. (F) Histological analysis of gd14.5 feto-placental labyrinth stained against CD31. (G) Quantification of feto-placental labyrinth CD31 staining in 3A (SPF n=6, GF n=6, ABX n=8) normalized to labyrinth area. (H) μCT scans of feto-placental arterial network, pseudocolored by vessel diameter. (I) Quantification of arterial volume from μCT scans in 3 C (SPF n=9, GF n=10, ABX n=5). (J) Quantification of arterial surface area from μCT scans in 3 C (SPF n=9, GF n=10, ABX n=5). As seen through these panels, microbiome depletion reduces mid-gestation feto-placental vasculature.

FIG. 12: Microbiota depletion does not vascular growth factor or barrier-related transcript expression in the E14.5 placenta. Transcript expression in E14.5 SPF (n=7), GF (n=10) and ABX (n=10) placentae for VEGFA (A), PGF (B), ECadherin (C), Occludin (D), Cadherin-5 (E) and ZO-2 (F). Not shown: Histological analysis of ZO-1 immunoreactivity in E14.5 junctional zone of SPF, GF, ABX and GF-CONV E14.5 placentae; quantification of ZO-1 fluorescence; and ZO1 transcript relative abundance in SPF, GF, ABX and GF-CONV E14.5 placentae.

FIG. 13: The maternal microbiota modulates the maternal and fetal sera metabolomes. Venn diagrams demonstrating significantly altered metabolites (black text), both downregulated (red text) and upregulated (blue text) in SPF (n=6), GF (n=6) and ABX (n=6) maternal serum (A) and fetal serum (B). Principle component analyses of maternal serum (C) and fetal serum (D) shown in (A-B). Truncated global metabolite profile changes in GF maternal serum relative to SPF (E), and ABX maternal serum relative to SPF (F). Truncated global metabolite profile changes in GF fetal serum relative to SPF (G), and ABX fetal serum relative to SPF (H). Top thirty metabolites defined by random forest analysis of maternal serum (I) and fetal serum (J).

FIG. 14: The maternal microbiome regulates metabolic subpathways in maternal and fetal sera. (A) Subpathway fold-enrichment analysis of GF and ABX maternal serum samples shown in FIG. 13A. (B) Subpathway fold-enrichment analysis of GF and ABX fetal serum samples shown in FIG. 13B.

FIG. 15: The maternal microbiome is a critical regulator of placental homeostasis and fetal development.

FIG. 16: Candidate bacterial taxa associated with reduced placental and fetal growth deficits revealed by 16S sequencing. (A) Alpha rarefaction plots demonstrating alpha-diversity based on observed OTUs from 16S sequencing of SPF, Abs and Non-Abs E14.5 fecal microbiota. (B) Weighted UniFrac analysis demonstrating beta-diversity of SPF (n=3), Abs (n=5) and Non-Abs (n=5) E14.5 fecal microbiota communities represented in (A). (C) Taxa bar plots demonstrating relative sequence abundance of bacterial taxa from SPF, Abs and Non-Abs E14.5 fecal microbiota communities represented in (A). (D) Relative abundance of family Peptostreptococcaceae in Abs and Non-Abs E14.5 fecal microbiota communities represented in (A). (E) Relative abundance of genus Enterococcus in Abs and Non-Abs E14.5 fecal microbiota communities represented in (A).

FIG. 17: Bacterial SCFA supplementation restores placental weight and microvascular impairments following maternal microbiota depletion. A) Schematic representation of SCFA administration to ABX dams. B) E14.5 placental weight by litter average for SPF (same as FIG. 1A), ABX (same as FIG. 1A) and ABX+SCFA (n=6). C) E14.5 fetal weight by litter average for litters shown in FIG. 6B. D) Representative images of microvasculature staining panel against laminin, TER119 and DAPI. E) Quantification of fetal labyrinth laminin raw integrated density normalized to fetal labyrinth area for litters shown in FIG. 6A-6B.

### DETAILED DESCRIPTION

In certain embodiments, the methods of the present disclosure are directed to promoting healthy growth in an unborn baby (e.g., fetus). In certain embodiments, the method comprises administering to a maternal subject gestating an unborn baby (e.g., fetus) a composition as described herein. In certain embodiments, the method comprises administering to a female subject a composition as described herein. Preferably, the method results in the unborn baby (e.g., fetus) exhibiting a lesser degree of Fetal Growth Restriction (FGR) relative to an unborn baby (e.g., fetus) gestated by a similar maternal subject (e.g., a maternal subject having a similar or identical maternal microbiome) not receiving the composition. Preferably, the method results in an increase in one or more of fetal weight, fetal volume, fetal surface area, fetal brain volume, fetal ventricle volume, and fetal brain:body ratio, relative to an unborn baby (e.g., fetus) gestated by a similar maternal subject (e.g., a maternal subject having a similar or identical maternal microbiome) not receiving the composition.

In certain embodiments, the methods of the present disclosure are directed to promoting placental development in a maternal subject gestating an unborn baby (e.g., fetus). In certain embodiments, the method comprises administering to the maternal subject a composition as described herein. In certain embodiments, the method comprises administering to a female subject a composition as described herein. Preferably, the method results in an increase in one or more of placental angiogenesis, placental immunity, and placental weight, relative to an unborn baby (e.g., fetus) gestated by a similar maternal subject (e.g., a maternal subject having a similar or identical maternal microbiome) not receiving the composition.

In certain embodiments, the methods of the present disclosure are directed to promoting angiogenesis in a fetal subject. In certain embodiments, the method comprises administering to a maternal subject gestating the unborn baby (e.g., fetus) a composition as described herein. In certain embodiments, the method comprises administering to a female subject a composition as described herein. Preferably, the method results in increased levels of uterine natural killer (uNK) cells.

In certain embodiments, the methods of the present disclosure are directed to inhibiting development of preeclampsia or Fetal Growth Restriction (FGR). In certain embodiments, the method comprises administering to a maternal subject gestating the unborn baby (e.g., fetus) a composition as described herein. In certain embodiments, the method comprises administering to a female subject a composition as described herein.

In certain embodiments, the methods further comprise administering the composition to the maternal subject or the female subject prior to gestation.

In certain embodiments, the female subject is a fertile, ovulating female subject. In certain embodiments, the female subject is a female subject seeking to implant an embryo.

In certain embodiments, the composition comprises a compound selected from primary bile acids, secondary bile acids, daidzein, genistein, osteopontin, osteoglycin, pleiotrophin, tyrosine, histidine, peptides, benzoate, tryptophan, 2-aminophenol sulfate, catechol sulfate, 4-hydroxyphenylacetate sulfate, N,N,N-trimethyl-5-aminovalerate, phenol sulfate, equol sulfate, hippurate, p-cresol sulfate, imidazolepropionate, indolepropionate, equol glucuronide, valerylglycine, phenol glucuronide, salicylate, S-carboxymethyl-L-cysteine, and trimethylamine N-oxide, and metabolites and derivatives thereof, or a combination thereof. In certain embodiments, the composition comprises a compound selected from primary bile acids, secondary bile acids, daidzein, genistein, tyrosine, histidine, peptides, benzoate, tryptophan, 2-aminophenol sulfate, catechol sulfate, 4-hydroxyphenylacetate sulfate, N,N,N-trimethyl-5-aminovalerate, phenol sulfate, equol sulfate, hippurate, p-cresol sulfate, imidazolepropionate, indolepropionate, equol glucuronide, valerylglycine, phenol glucuronide, salicylate, S-carboxymethyl-L-cysteine, and trimethylamine N-oxide, and metabolites and derivatives thereof, or a combination thereof. In certain embodiments, the composition comprises a compound selected from primary bile acids, secondary bile acids, daidzein, genistein, tyrosine, histidine, benzoate, tryptophan, 2-aminophenol sulfate, catechol sulfate, 4-hydroxyphenylacetate sulfate, N,N,N-trimethyl-5-aminovalerate, phenol sulfate, equol sulfate, hippurate, p-cresol sulfate, imidazolepropionate, indolepropionate, equol glucuronide, valerylglycine, phenol glucuronide, salicylate, S-carboxymethyl-L-cysteine, and trimethylamine N-oxide, and metabolites and derivatives thereof, or a combination thereof. Suitable derivatives include, but are not limited to, acetylated compounds and glucuronides and prodrugs of the compounds.

Daidzein has the following chemical structure:

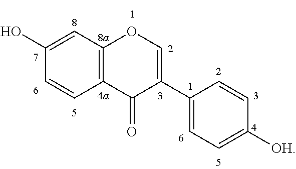 [SMILES: O=c1c(-c2ccc(O)cc2)coc2cc(O)ccc12]

Genistein has the following chemical structure:

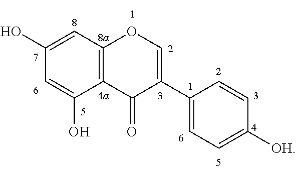 [SMILES: O=c1c(-c2ccc(O)cc2)coc2cc(O)cc(O)c12]

Suitable derivatives of daidzein and genistein include, but are not limited to, glucuronides and prodrugs of the compounds.

Bile acids are steroid acids found predominantly in the bile of mammals and other vertebrates. Different molecular forms of bile acids can be synthesized in the liver by different species. Bile acids are conjugated with taurine or glycine in the liver, and the sodium and potassium salts of these conjugated bile acids are called bile salts. Primary bile acids are those synthesized by the liver. Secondary bile acids result from bacterial actions in the colon. In humans, taurocholic acid and glycocholic acid (derivatives of cholic acid) and taurochenodeoxycholic acid and glycochenodeoxycholic acid (derivatives of chenodeoxycholic acid) are the major bile salts in bile and are roughly equal in concentration. The conjugated salts of their 7-alpha-dehydroxylated derivatives, deoxycholic acid and lithocholic acid, are also found, as are derivatives of cholic, chenodeoxycholic and deoxycholic acids. Suitable primary and secondary bile acids include, but are not limited to, the foregoing and others known in the art.

Osteopontin is an extracellular structural protein expressed in bone, and it has roles in biomineralization, bone remodeling, and immune functions, including heart conditions, chemotaxis, cell activation, and apoptosis. Osteopontin is biosynthesized by a variety of tissue types, including cardiac fibroblasts, preosteoblasts, osteoblasts, osteocytes, odontoblasts, some bone marrow cells, hypertrophic chondrocytes, dendritic cells, macrophages, smooth muscle, skeletal muscle myoblasts, endothelial cells, and extraosseous (non-bone) cells in the inner ear, brain, kidney, deciduum, and placenta. Synthesis of osteopontin is stimulated by calcitriol (1,25-dihydroxy-vitamin D₃).

Osteoglycin is a human protein which induces ectopic bone formation in conjunction with transforming growth factor beta. The level of expression of the OGN gene, which encodes osteoglycin, has been correlated with enlarged hearts, specifically left ventricular hypertrophy.

Pleiotrophin is a growth factor in humans with a high affinity for heparin. During embryonic and early postnatal development, pleiotrophin is expressed in the central and peripheral nervous system and also in several non-neural tissues, including the lung, kidney, gut, and bone. In the adult central nervous system, pleiotrophin is expressed in an activity-dependent manner in the hippocampus, where it can suppress long-term potentiation induction.

“Microbiome,” as used herein, refers to the microorganisms in a given environment, such as the body or a part of the body. The “maternal microbiome,” as used herein, refers to the microorganisms in a maternal subject (i.e., a pregnant or gestating subject), particularly in the gut of the maternal subject. The gut microbiome modulates the bioavailability of hundreds of biochemicals in the circulating blood. During pregnancy, the maternal gut environment supplies nutrients and growth factors, from the maternal diet and other nutritional intake, to nurture offspring growth.

A “depleted” maternal microbiome is characterized by a reduced level of one or more microbial species (e.g., one or more bacterial species), such as 95%, 90%, 85%, 80%, 75%, 70%, 65%, 60%, 55%, 50%, 45%, 40%, 35%, 30%, 25%, 20%, 15%, 10%, 5%, 4%, 3%, 2%, 1%, 0.5%, or 0.1% of the level relative to a maternal subject without a depleted maternal microbiome.

“Germ-free” (GF) subjects, as used herein, are subjects with no microorganisms living in or on them. “Antibiotic-treated” (ABX) subjects, as used herein, are subjects treated with one or more antibiotic compounds, many representative examples of which are known in the art.

In some embodiments, the present invention is drawn to a composition comprising at least one bacterial species or bacterial strain (e.g., a probiotic bacterial strain) capable of promoting fetal growth, placental development, and/or angiogenesis, optionally wherein the at least one bacterial species or bacterial strain is alive and capable of proliferation. Such bacteria (e.g., probiotic bacteria) inhibit one or more adverse effects of maternal microbiota depletion (e.g., in ABX subjects) on fetal, placental, or angiogenic development, e.g., restrictions in fetal weight, fetal volume, fetal surface area, fetal brain volume, fetal ventricle volume, fetal brain:body ratio, placental angiogenesis, placental immunity, placental weight, and/or levels of uNK cells. In some embodiments, such bacteria restore expression of one or more genes relevant to fetal, placental, or angiogenic development.

### Definitions

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); and/or mammals, including commercially relevant mammals such as cattle, pigs, horses, sheep, goats, cats, and/or dogs. Preferred subjects are humans.

An “ovulating” female subject, as used herein, refers to a female subject having a regular cycle of menses, e.g., a female between menarche and menopause that is not employing hormonal birth control that inhibits ovulation. A “fertile” female subject, as used herein, refers to an ovulating female subject able to conceive offspring.

As used herein, a therapeutic that “prevents” a disorder or condition refers to a compound or composition that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the subject of one or more of the disclosed compositions. If it is administered prior to clinical manifestation of the unwanted condition (e.g., disease or other unwanted state of the subject) then the treatment is prophylactic (i.e., it protects the subject against developing the unwanted condition), whereas if it is administered after manifestation of the unwanted condition, the treatment is therapeutic (i.e., it is intended to diminish, ameliorate, or stabilize the existing unwanted condition or side effects thereof).

The term “prodrug” is intended to encompass compounds which, under physiologic conditions, are converted into therapeutically active agents. A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) and esters or amides of phosphates and phosphonic acids are preferred prodrugs of the present invention.

As used herein, the term “about” is defined as being close to as understood by one of ordinary skill in the art. In one non-limiting embodiment, the term “about” is defined to be within 10%, preferably within 5%, more preferably within 1%, and most preferably within 0.5%.

### Bacterial Compositions

In certain aspects, provided herein are bacterial compositions comprising a spore-forming bacteria and optionally a compound selected from primary bile acids, secondary bile acids, daidzein, genistein, osteopontin, osteoglycin, pleiotrophin, tyrosine, histidine, peptides, benzoate, tryptophan, 2-aminophenol sulfate, catechol sulfate, 4-hydroxyphenylacetate sulfate, N,N,N-trimethyl-5-aminovalerate, phenol sulfate, equol sulfate, hippurate, p-cresol sulfate, imidazolepropionate, indolepropionate, equol glucuronide, valerylglycine, phenol glucuronide, salicylate, S-carboxymethyl-L-cysteine, and trimethylamine N-oxide, and metabolites and derivatives thereof. In certain aspects, provided herein are bacterial compositions comprising a spore-forming bacteria and optionally a compound selected from primary bile acids, secondary bile acids, daidzein, genistein, tyrosine, histidine, peptides, benzoate, tryptophan, 2-aminophenol sulfate, catechol sulfate, 4-hydroxyphenylacetate sulfate, N,N,N-trimethyl-5-aminovalerate, phenol sulfate, equol sulfate, hippurate, p-cresol sulfate, imidazolepropionate, indolepropionate, equol glucuronide, valerylglycine, phenol glucuronide, salicylate, S-carboxymethyl-L-cysteine, and trimethylamine N-oxide, and metabolites and derivatives thereof. Preferably, the spore-forming bacterium is of a bacterial species found in the maternal microbiome (e.g., the maternal gut microbiome), including, but not limited to, a bacterial species selected from phylum Firmicutes (such as Clostridia), phylum Bacteroidetes (such as genus Bacteroides (e.g., B. thetaiotaomicron, B. uniformis, B. vulgatus, B. ovatus, B. fragilus)), phylum Proteobacteria (such as family Enterobacteriaecea), or a combination thereof. In some embodiments, the bacterial formulation comprises a bacterium and/or a combination of bacteria described herein and a pharmaceutically acceptable carrier.

In certain embodiments, at least 50%, 55%, 60%, 65%, 70%, 75%, 80%, 85%, 90%, 91%, 92%, 93%, 94%, 95%, 96%, 97%, 98% or 99% of the bacteria in the bacterial composition are phylum Firmicutes, phylum Bacteroidetes, phylum Proteobacteria, or a combination thereof. In certain embodiments, substantially all of the bacteria in the bacterial composition are phylum Firmicutes, phylum Bacteroidetes, phylum Proteobacteria, or a combination thereof. In certain embodiments, the bacterial composition comprises at least 1×10³ colony forming units (CFUs), 1×10⁴ colony forming units (CFUs), 1×10⁵ colony forming units (CFUs), 5×10⁵ colony forming units (CFUs), 1×10⁶ colony forming units (CFUs), 2×10⁶ colony forming units (CFUs), 3×10⁶ colony forming units (CFUs), 4×10⁶ colony forming units (CFUs), 5×10⁶ colony forming units (CFUs), 6×10⁶ colony forming units (CFUs), 7×10⁶ colony forming units (CFUs), 8×10⁶ colony forming units (CFUs), 9×10⁶ colony forming units (CFUs), 1×10⁷ colony forming units (CFUs), 2×10⁷ colony forming units (CFUs), 3×10⁷ colony forming units (CFUs), 4×10⁷ colony forming units (CFUs), 5×10⁷ colony forming units (CFUs), 6×10⁷ colony forming units (CFUs), 7×10⁷ colony forming units (CFUs), 8×10⁷ colony forming units (CFUs), 9×10⁷ colony forming units (CFUs), 1×10⁸ colony forming units (CFUs), 2×10⁸ colony forming units (CFUs), 3×10⁸ colony forming units (CFUs), 4×10⁸ colony forming units (CFUs), 5×10⁸ colony forming units (CFUs), 6×10⁸ colony forming units (CFUs), 7×10⁸ colony forming units (CFUs), 8×10⁸ colony forming units (CFUs), 9×10⁸ colony forming units (CFUs), 1×10⁹ colony forming units (CFUs), 5×10⁹ colony forming units (CFUs), 1×10¹⁰ colony forming units (CFUs) 5×10¹⁰ colony forming units (CFUs), 1×1011 colony forming units (CFUs) 5×1011 colony forming units (CFUs), 1×10¹² colony forming units (CFUs) 5×10¹² colony forming units (CFUs), 1×10¹³ colony forming units (CFUs) of phylum Firmicutes, phylum Bacteroidetes, phylum Proteobacteria, or a combination thereof.

The selected dosage level will depend upon a variety of factors including the subject's diet, the route of administration, the time of administration, the residence time of the particular microorganism being employed, the duration of the treatment, other drugs, compounds and/or materials used in combination with the particular composition employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the bacteria employed in the pharmaceutical composition at levels lower than that required in order to achieve the desired therapeutic effect and gradually increase the dosage until the desired effect is achieved.

In some embodiments, probiotic formulations containing phylum Firmicutes, phylum Bacteroidetes, phylum Proteobacteria, or a combination thereofs are provided as encapsulated, enteric coated, or powder forms, with doses ranging up to 10¹¹ cfu (e.g., up to 10¹⁰ cfu). In some embodiments, the composition comprises 5×10¹¹ cfu of phylum Firmicutes, phylum Bacteroidetes, phylum Proteobacteria, or a combination thereof and 10% (w/w) corn starch in a capsule. In some embodiments, the capsule is enteric coated, e.g., for duodenal release at pH 5.5. In some embodiments, the composition comprises a powder of freeze-dried phylum Firmicutes, phylum Bacteroidetes, phylum Proteobacteria, or a combination thereof which is deemed to have “Qualified Presumption of Safety” (QPS) status. In some embodiments, the composition is storage-stable at frozen or refrigerated temperature. As used herein, “stably stored” or “storage-stable” refer to a composition in which cells are able to withstand storage for extended periods of time (e.g., at least one month, or two, three, four, six, or twelve months or more) with a less than 95%, 90%, 80%, 70%, 60%, 50%, 40%, 30%, 20%, 15%, 10%, 5%, or 1% decrease in cell viability.

Methods for producing microbial compositions may include three main processing steps. The steps are: organism banking, organism production, and preservation. In certain embodiments, a sample that contains an abundance of phylum Firmicutes, phylum Bacteroidetes, phylum Proteobacteria, or a combination thereof may be cultured by avoiding an isolation step.

For banking, phylum Firmicutes, phylum Bacteroidetes, phylum Proteobacteria, or a combination thereof included in the microbial composition may be (1) isolated directly from a specimen or taken from a banked stock, (2) optionally cultured on a nutrient agar or broth that supports growth to generate viable biomass, and (3) the biomass optionally preserved in multiple aliquots in long-term storage.

In embodiments using a culturing step, the agar or broth may contain nutrients that provide essential elements and specific factors that enable growth. An example would be a medium composed of 20 g/L glucose, 10 g/L yeast extract, 10 g/L soy peptone, 2 g/L citric acid, 1.5 g/L sodium phosphate monobasic, 100 mg/L ferric ammonium citrate, 80 mg/L magnesium sulfate, 10 mg/L hemin chloride, 2 mg/L calcium chloride, 1 mg/L menadione. Another example would be a medium composed of 10 g/L beef extract, 10 g/L peptone, 5 g/L sodium chloride, 5 g/L dextrose, 3 g/L yeast extract, 3 g/L sodium acetate, 1 g/L soluble starch, and 0.5 g/L L-cysteine HCl, at pH 6.8. A variety of microbiological media and variations are well known in the art (e.g., R. M. Atlas, Handbook of Microbiological Media (2010) CRC Press). Culture media can be added to the culture at the start, may be added during the culture, or may be intermittently/continuously flowed through the culture. The strains in the bacterial composition may be cultivated alone, as a subset of the microbial composition, or as an entire collection comprising the microbial composition. As an example, a first strain may be cultivated together with a second strain in a mixed continuous culture, at a dilution rate lower than the maximum growth rate of either cell to prevent the culture from washing out of the cultivation.

The inoculated culture is incubated under favorable conditions for a time sufficient to build biomass. For microbial compositions for human use this is often at 37° C. temperature, pH, and other parameter with values similar to the normal human niche. The environment may be actively controlled, passively controlled (e.g., via buffers), or allowed to drift. For example, for anaerobic bacterial compositions, an anoxic/reducing environment may be employed. This can be accomplished by addition of reducing agents such as cysteine to the broth, and/or stripping it of oxygen. As an example, a culture of a bacterial composition may be grown at 37° C., pH 7, in the medium above, pre-reduced with 1 g/L cysteine-HCl.

When the culture has generated sufficient biomass, it may be preserved for banking. The organisms may be placed into a chemical milieu that protects from freezing (adding ‘cryoprotectants’), drying (‘lyoprotectants’), and/or osmotic shock (‘osmoprotectants’), dispensing into multiple (optionally identical) containers to create a uniform bank, and then treating the culture for preservation. Containers are generally impermeable and have closures that assure isolation from the environment. Cryopreservation treatment is accomplished by freezing a liquid at ultra-low temperatures (e.g., at or below −80° C.). Dried preservation removes water from the culture by evaporation (in the case of spray drying or ‘cool drying’) or by sublimation (e.g., for freeze drying, spray freeze drying). Removal of water improves long-term microbial composition storage stability at temperatures elevated above cryogenic conditions. Microbial composition banking may be done by culturing and preserving the strains individually, or by mixing the strains together to create a combined bank. As an example of cryopreservation, a microbial composition culture may be harvested by centrifugation to pellet the cells from the culture medium, the supernatant decanted and replaced with fresh culture broth containing 15% glycerol. The culture can then be aliquoted into 1 mL cryotubes, sealed, and placed at −80° C. for long-term viability retention. This procedure achieves acceptable viability upon recovery from frozen storage.

Microbial production may be conducted using similar culture steps to banking, including medium composition and culture conditions described above. It may be conducted at larger scales of operation, especially for clinical development or commercial production. At larger scales, there may be several subcultivations of the microbial composition prior to the final cultivation. At the end of cultivation, the culture is harvested to enable further formulation into a dosage form for administration. This can involve concentration, removal of undesirable medium components, and/or introduction into a chemical milieu that preserves the microbial composition and renders it acceptable for administration via the chosen route. For example, a microbial composition may be cultivated to a concentration of 10¹⁰ CFU/mL, then concentrated 20-fold by tangential flow microfiltration; the spent medium may be exchanged by diafiltering with a preservative medium consisting of 2% gelatin, 100 mM trehalose, and 10 mM sodium phosphate buffer. The suspension can then be freeze-dried to a powder and titrated.

After drying, the powder may be blended to an appropriate potency, and mixed with other cultures and/or a filler such as microcrystalline cellulose for consistency and ease of handling, and the bacterial composition formulated as provided herein.

In certain aspects, provided are bacterial compositions for administration in subjects. In some embodiments, the bacterial compositions are combined with additional active and/or inactive materials in order to produce a final product, which may be in single dosage unit or in a multi-dose format.

In some embodiments, the composition comprises at least one carbohydrate. A “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). Carbohydrates may exist in many different forms, for example, conformers, cyclic forms, acyclic forms, stereoisomers, tautomers, anomers, and isomers.

In some embodiments, the composition comprises at least one lipid. As used herein, a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans). In some embodiments the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0). In some embodiments the composition comprises at least one modified lipid, for example a lipid that has been modified by cooking.

In some embodiments, the composition comprises at least one supplemental mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In some embodiments, the composition comprises at least one supplemental vitamin. The at least one vitamin can be fat-soluble or water soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In some embodiments, the composition comprises an excipient. Non-limiting examples of suitable excipients include a buffering agent, a preservative, a stabilizer, a binder, a compaction agent, a lubricant, a dispersion enhancer, a disintegration agent, a flavoring agent, a sweetener, and a coloring agent.

In some embodiments, the excipient is a buffering agent. Non-limiting examples of suitable buffering agents include sodium citrate, magnesium carbonate, magnesium bicarbonate, calcium carbonate, and calcium bicarbonate.

In some embodiments, the excipient comprises a preservative. Non-limiting examples of suitable preservatives include antioxidants, such as alpha-tocopherol and ascorbate, and antimicrobials, such as parabens, chlorobutanol, and phenol.

In some embodiments, the composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

In some embodiments, the composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, the composition comprises a dispersion enhancer as an excipient. Non-limiting examples of suitable dispersants include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments, the compositions of the present invention are combined with a carrier (e.g., a pharmaceutically acceptable carrier) which is physiologically compatible with the gastrointestinal tissue of the subject(s) to which it is administered. Carriers can be comprised of solid-based, dry materials for formulation into tablet, capsule or powdered form; or the carrier can be comprised of liquid or gel-based materials for formulations into liquid or gel forms. The specific type of carrier, as well as the final formulation depends, in part, upon the selected route(s) of administration. The therapeutic composition of the present invention may also include a variety of carriers and/or binders. In some embodiments, the carrier is micro-crystalline cellulose (MCC) added in an amount sufficient to complete the one gram dosage total weight. Carriers can be solid-based dry materials for formulations in tablet, capsule or powdered form, and can be liquid or gel-based materials for formulations in liquid or gel forms, which forms depend, in part, upon the routes of administration. Typical carriers for dry formulations include, but are not limited to: trehalose, malto-dextrin, rice flour, microcrystalline cellulose (MCC) magnesium sterate, inositol, FOS, GOS, dextrose, sucrose, and like carriers. Suitable liquid or gel-based carriers include but are not limited to: water and physiological salt solutions; urea; alcohols and derivatives (e.g., methanol, ethanol, propanol, butanol); glycols (e.g., ethylene glycol, propylene glycol, and the like). Preferably, water-based carriers possess a neutral pH value (i.e., approximately pH 7.0). Other carriers or agents for administering the compositions described herein are known in the art, e.g., in U.S. Pat. No. 6,461,607.

In some embodiments, the composition comprises a disintegrant as an excipient. In some embodiments the disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some embodiments the disintegrant is an effervescent disintegrant. Non-limiting examples of suitable effervescent disintegrants include sodium bicarbonate in combination with citric acid, and sodium bicarbonate in combination with tartaric acid.

In some embodiments, the bacterial formulation comprises an enteric coating or micro encapsulation. In certain embodiments, the enteric coating or micro encapsulation improves targeting to a desired region of the gastrointestinal tract. For example, in certain embodiments, the bacterial composition comprises an enteric coating and/or microcapsules that dissolves at a pH associated with a particular region of the gastrointestinal tract. In some embodiments, the enteric coating and/or microcapsules dissolve at a pH of about 5.5-6.2 to release in the duodenum, at a pH value of about 7.2-7.5 to release in the ileum, and/or at a pH value of about 5.6-6.2 to release in the colon. Exemplary enteric coatings and microcapsules are described, for example, in U.S. Pat. Pub. No. 2016/0022592, which is hereby incorporated by reference in its entirety.

In some embodiments, the composition is a food product (e.g., a food or beverage) such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Specific examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, and Chinese soups; soups; dairy products such as milk, dairy beverages, ice creams, cheeses, and yogurts; fermented products such as fermented soybean pastes, yogurts, fermented beverages, and pickles; bean products; various confectionery products, including biscuits, cookies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies. The composition may be a fermented food product, such as, but not limited to, a fermented milk product. Non-limiting examples of fermented food products include kombucha, sauerkraut, pickles, miso, tempeh, natto, kimchi, raw cheese, and yogurt. The composition may also be a food additive, such as, but not limited to, an acidulent (e.g., vinegar). Food additives can be divided into several groups based on their effects. Non-limiting examples of food additives include acidulents (e.g., vinegar, citric acid, tartaric acid, malic acid, fumaric acid, and lactic acid), acidity regulators, anticaking agents, antifoaming agents, foaming agents, antioxidants (e.g., vitamin C), bulking agents (e.g., starch), food coloring, fortifying agents, color retention agents, emulsifiers, flavors and flavor enhancers (e.g., monosodium glutamate), flour treatment agents, glazing agents, humectants, tracer gas, preservatives, stabilizers, sweeteners, and thickeners.

In certain embodiments, the bacteria disclosed herein are administered in conjunction with a prebiotic to the subject. Prebiotics are carbohydrates which are generally indigestible by a host animal and are selectively fermented or metabolized by bacteria. Prebiotics may be short-chain carbohydrates (e.g., oligosaccharides) and/or simple sugars (e.g., mono- and di-saccharides) and/or mucins (heavily glycosylated proteins) that alter the composition or metabolism of a microbiome in the host. The short chain carbohydrates are also referred to as oligosaccharides, and usually contain from 2 or 3 and up to 8, 9, 10, 15 or more sugar moieties. When prebiotics are introduced to a host, the prebiotics affect the bacteria within the host and do not directly affect the host. In certain aspects, a prebiotic composition can selectively stimulate the growth and/or activity of one of a limited number of bacteria in a host. Prebiotics include oligosaccharides such as fructooligosaccharides (FOS) (including inulin), galactooligosaccharides (GOS), trans-galactooligosaccharides, xylooligosaccharides (XOS), chitooligosaccharides (COS), soy oligosaccharides (e.g., stachyose and raffinose) gentiooligosaccharides, isomaltooligosaccharides, mannooligosaccharides, maltooligosaccharides and mannanoligosaccharides. Oligosaccharides are not necessarily single components, and can be mixtures containing oligosaccharides with different degrees of oligomerization, sometimes including the parent disaccharide and the monomeric sugars. Various types of oligosaccharides are found as natural components in many common foods, including fruits, vegetables, milk, and honey. Specific examples of oligosaccharides are lactulose, lactosucrose, palatinose, glycosyl sucrose, guar gum, gum Arabic, tagalose, amylose, amylopectin, pectin, xylan, and cyclodextrins. Prebiotics may also be purified or chemically or enzymatically synthesized.

### Pharmaceutical Compositions

The compositions and methods of the present invention may be utilized to treat a subject in need thereof. In certain embodiments, the subject is a mammal such as a human, or a non-human mammal. When administered to subject, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of the disclosed compounds. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, bitartaric, citric, ascorbic, maleic, benzoic, phenylacetic, cinnamic, salicylic, and sulfosalicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds disclosed herein are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds disclosed herein for laboratory use, or for subsequent conversion to a pharmaceutically acceptable acid addition salt.

The term “pharmaceutically acceptable basic addition salt” as used herein means any non-toxic organic or inorganic base addition salt of any acid compounds disclosed herein. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, intraocular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, about 0.1 to about 99.5% (more preferably, about 0.5 to about 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the subject's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the subject, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

In certain embodiments, conjoint administration of compounds of the invention with one or more additional therapeutic agent(s) provides improved efficacy relative to each individual administration of the compound of the invention or the one or more additional therapeutic agent(s). In certain such embodiments, the conjoint administration provides an additive effect, wherein an additive effect refers to the sum of each of the effects of individual administration of the compound of the invention and the one or more additional therapeutic agent(s).

This invention includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### EXAMPLES

### Example 1: Dissecting Interactions Between the Maternal Microbiome, Placental Immunity, and Angiogenesis

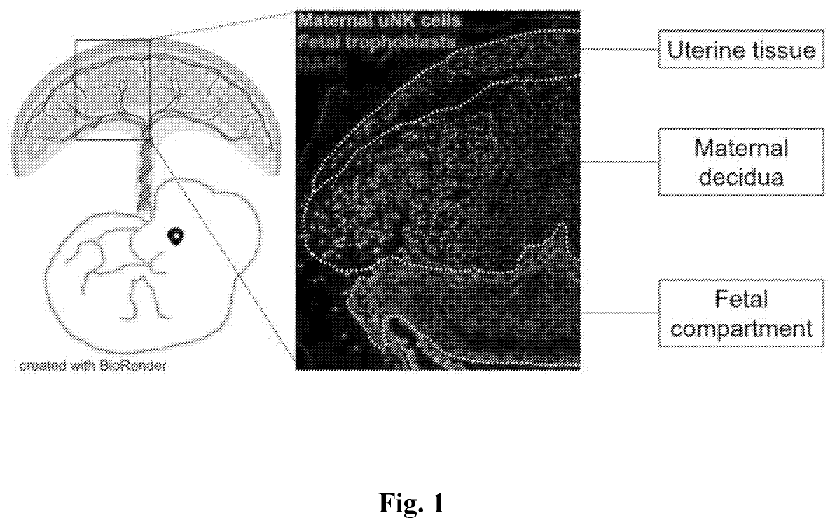

The placenta regulates maternal-fetal exchange, facilitating transport of biochemicals essential for fetal growth (FIG. 1). Placental insufficiencies occur in 3-8% of pregnancies worldwide, and are often comorbid with fetal intrauterine growth restriction. The maternal decidua is dominated by specialized uterine natural killer cells (uNKs) that promote vascular and fetal growth.

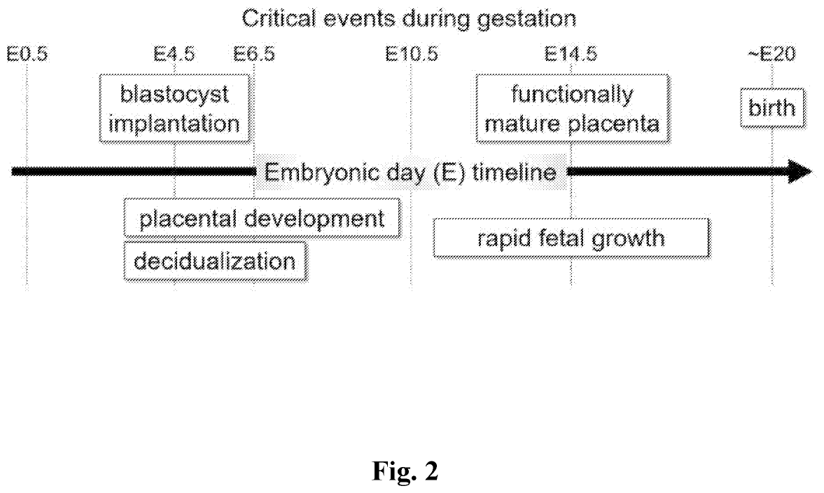

Some critical events during gestation are illustrated in FIG. 2. Experimental conditions that were explored included Specific pathogen-free (SPF); Germ-free (GF); broadspectrum antibiotic treatment (ABX); GF-conventionalized (GF-CONV).

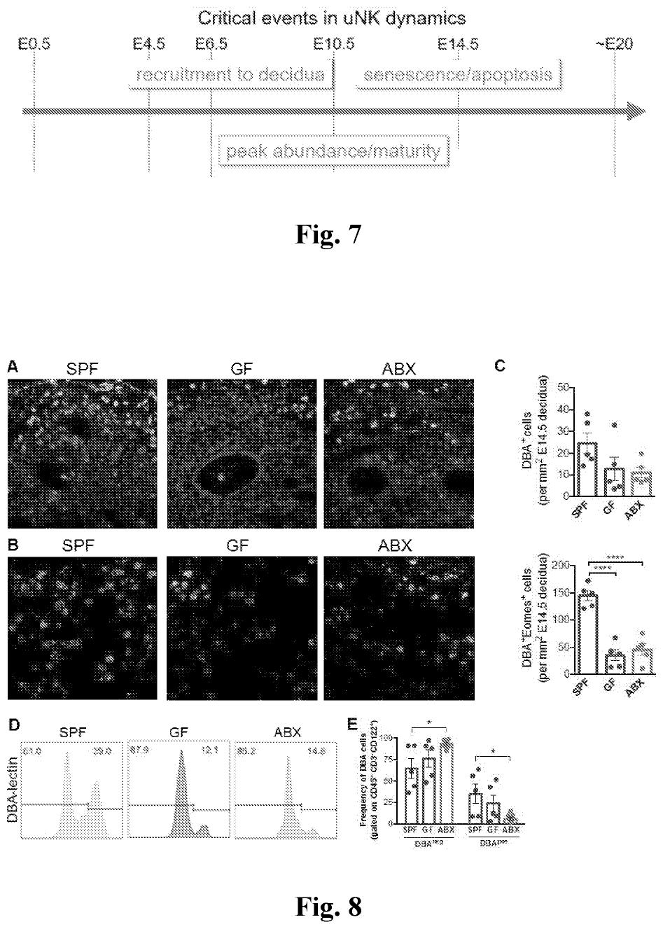

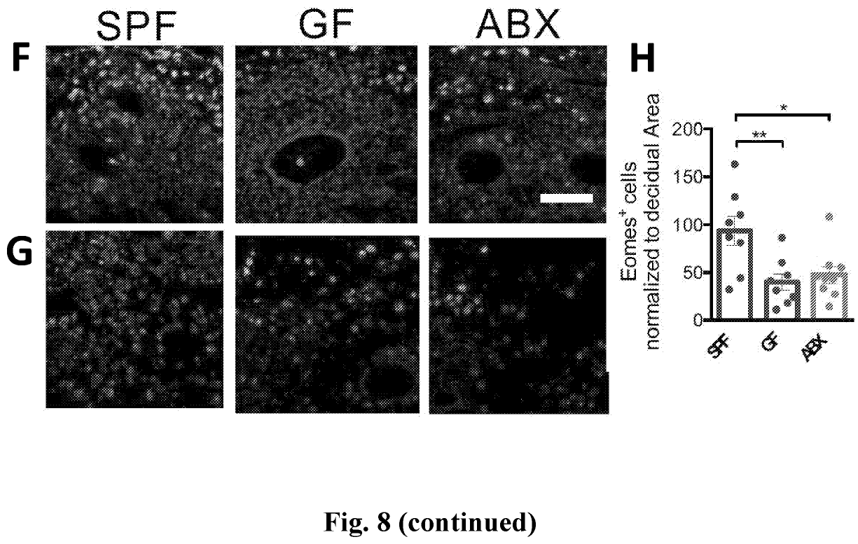

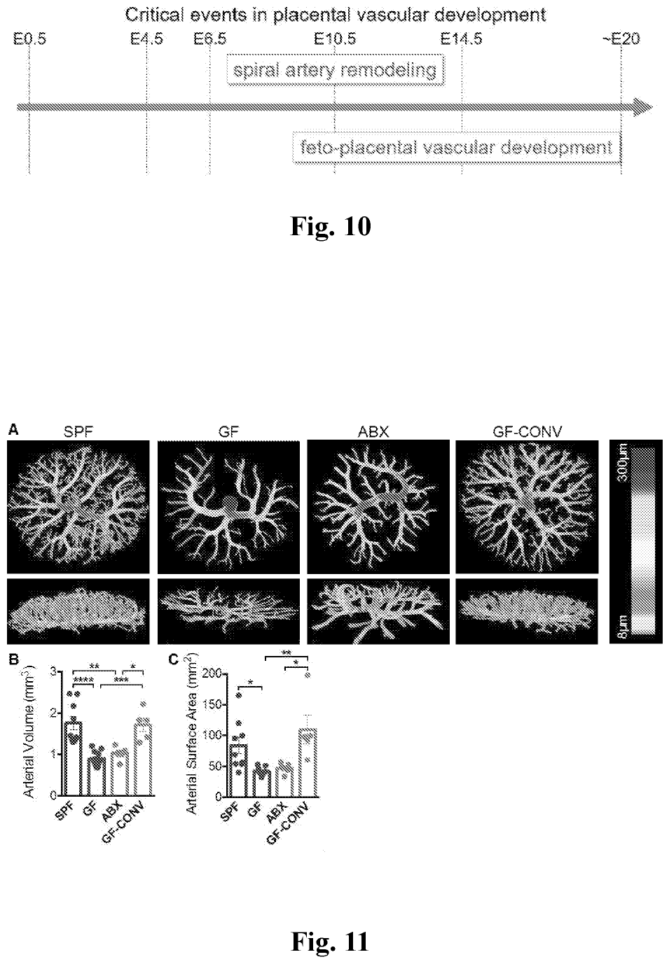

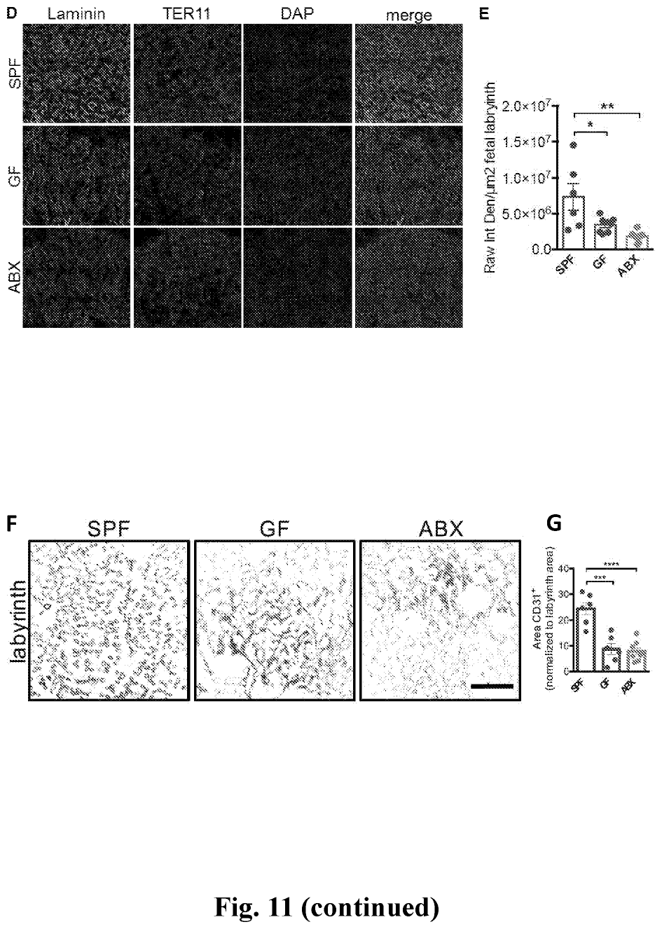

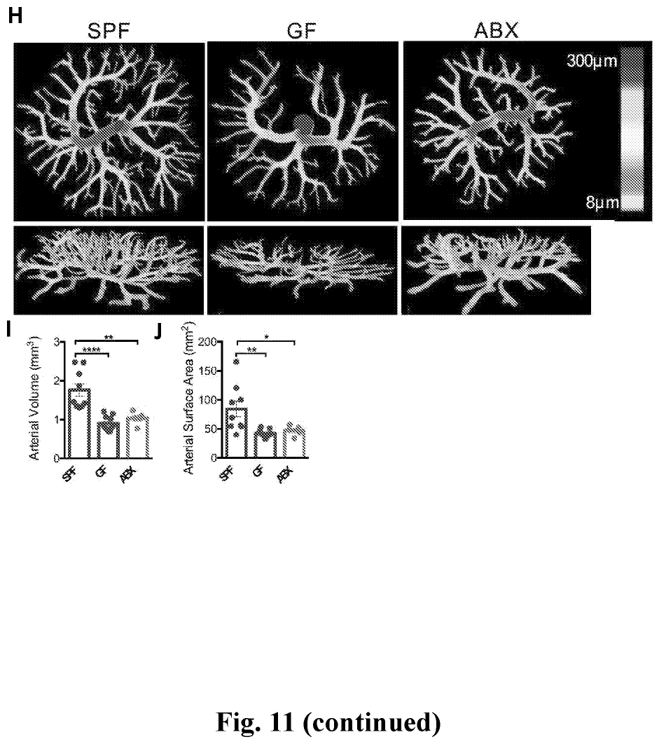

Our data show that the maternal microbiome regulates placental immune homeostasis (FIG. 7 and FIG. 8). In addition, our data show that the maternal microbiome regulates placental vascular development (FIG. 10 and FIG. 11). Further details are provided in Example 3.

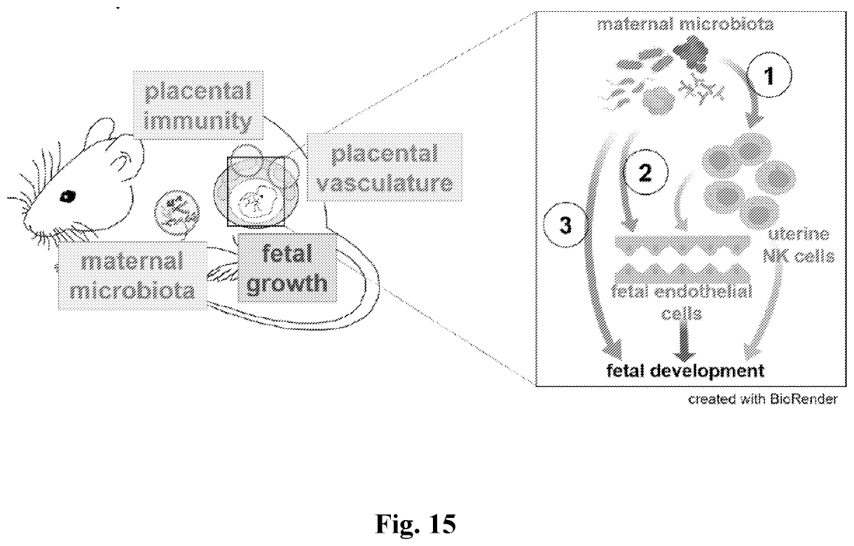

A working model is provided in FIG. 15, which illustrates that the maternal microbiome is a critical regulator of placental homeostasis and fetal development.

### Example 2: Dissecting Interactions Between the Maternal Microbiome, Placental Immunity, Angiogenesis, and Fetal Growth

### A1: Determining Whether the Maternal Microbiome Alters uNK Phenotype and Function

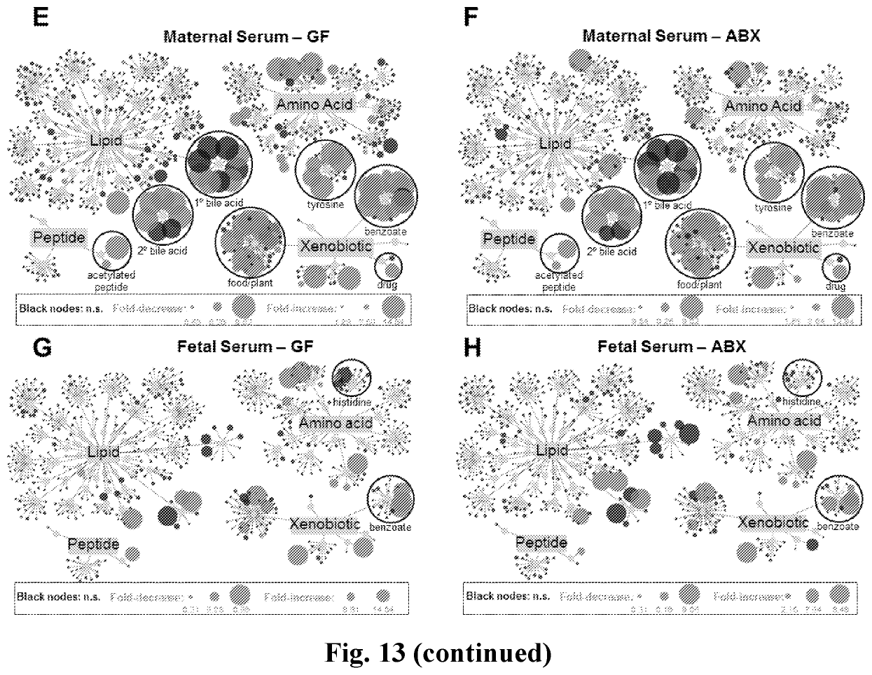

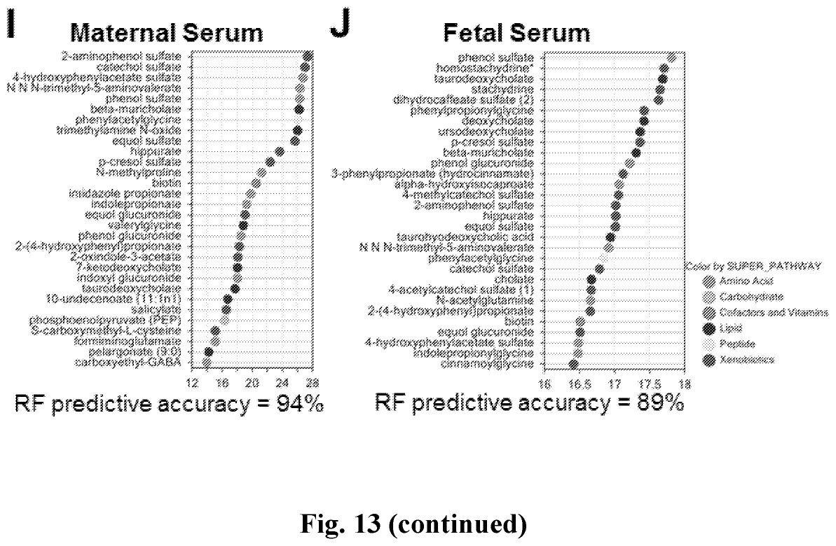

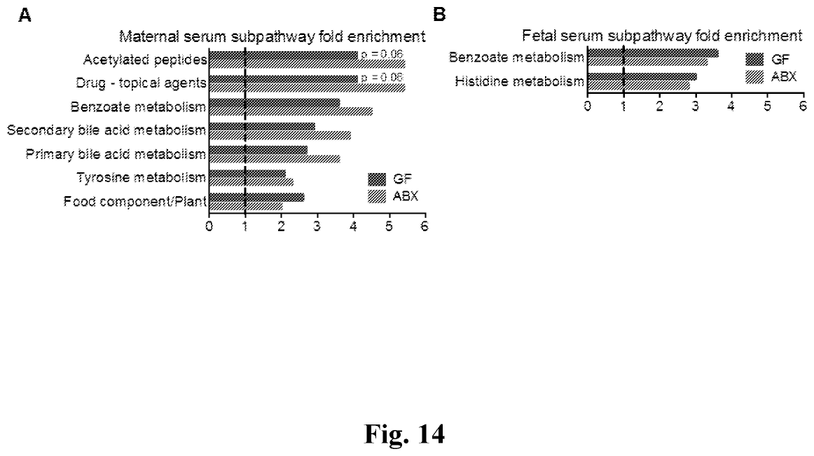

Data reveal that the maternal microbiome is responsible for regulating hundreds of biochemicals in the maternal blood (FIG. 13, FIG. 14). FIG. 13 shows mass-spectrometry based analysis of metabolites from gestational day (gd)14.5 SPF, GF and ABX maternal serum, and hundreds of differentially regulated metabolites were identified in GF and ABX mice relative to SPF controls. In addition, depleting the maternal microbiome reduces numbers of angiogenic uNKs at mid-gestation (FIG. 8). 1.1: To determine how the maternal microbiome influences uNK phenotypes, uNKs are profiled by flow cytometry and imaging of placentas from conventionally colonized (SPF), germ-free (GF), antibiotic-treated (ABX) and conventionalized (GF-CONV) dams. 1.2: To characterize the effects of the maternal microbiome on placental gene expression, transcriptional signatures of uNKs and other placental cells are profiled by single-cell RNA sequencing of placentas from SPF, GF, ABX and GF-CONV dams. 1.3: To assess whether the maternal microbiome influences uNK activity, uNKs from gnotobiotic mice and mice treated with microbial biochemicals are assessed and effector function is analyzed by flow cytometry, confocal imaging and in vitro assays.

### A2: Roles for the Maternal Microbiome in Regulating Placental Vascular Development

Data shows that depletion of the maternal microbiome reduces feto-placental vasculature (FIG. 11), coincident with reduced angiogenic uNKs. 2.1: To characterize effects of the maternal microbiome on feto-placental vasculature, vascular structure is analyzed by micro-computed tomography (μCT) and confocal imaging of placentas from SPF, GF, ABX and GF-CONV dams. 2.2: To identify microbial factors that impact vascular structure, microbiome-dependent metabolites are supplemented into gnotobiotic mice or in vitro functional assays and evaluate vasculature using methods described in 2.1. 2.3: To determine whether microbial effects on uNKs influence placental vascular structure, NKs are transferred from SPF mice into gnotobiotic littermates and placental vasculature is assessed as in 2.1.

### A3: Mechanistic Influences of the Maternal Microbiome on Fetal Growth

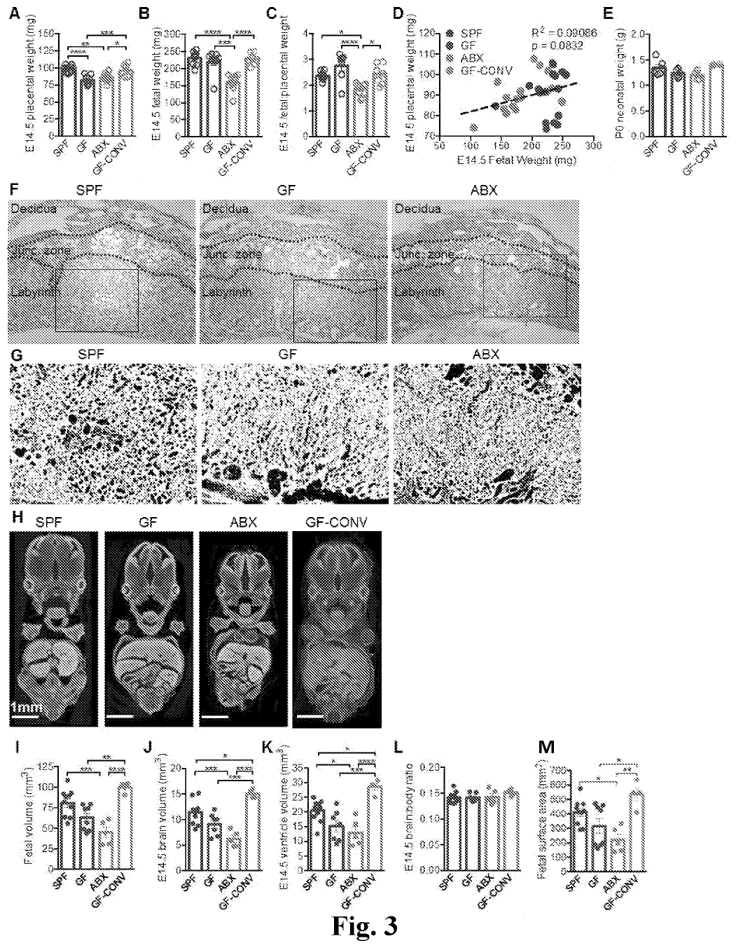

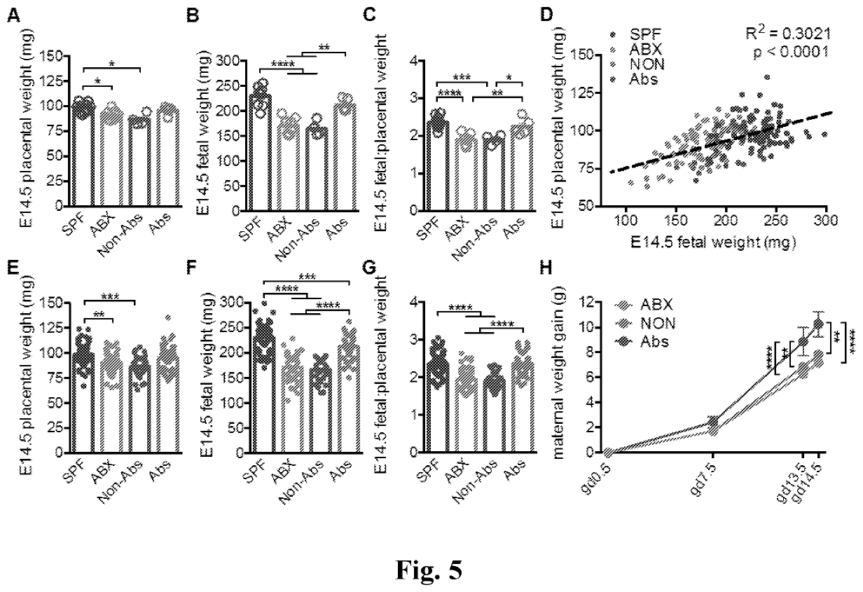

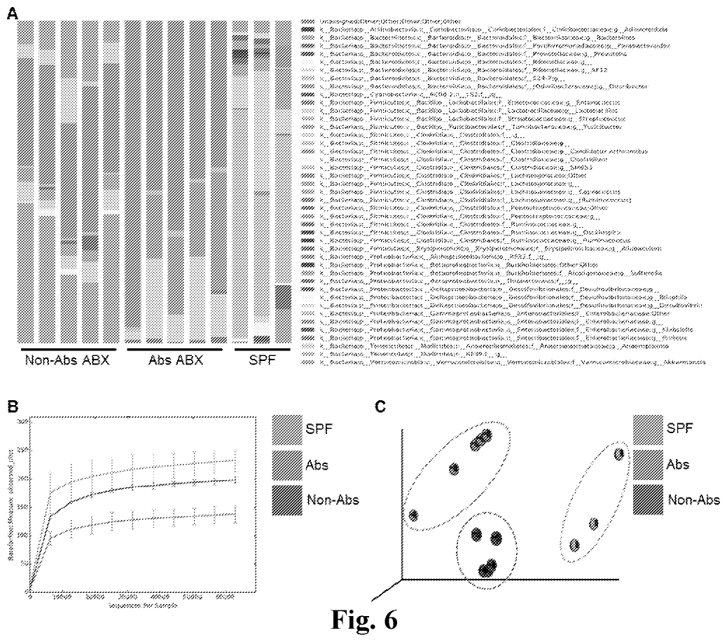

Maternal health is paramount for supporting normal fetal growth. FIG. 13 and FIG. 14 reveal important roles for the microbiome in regulating maternal circulating biochemicals, maternal placental immunity, feto-placental vasculature and fetal circulating metabolites, while FIG. 3 and FIG. 5 show that transient depletion of the maternal microbiota restricts fetal growth during gestation. 3.1: To test whether the maternal microbiome and microbial factors impact offspring development, fetal growth from gnotobiotic dams supplemented with select bacterial taxa or microbiome-dependent metabolites are assessed. 3.2: To interrogate roles for uNKs in mediating effects of the microbiome on fetal growth, SPF NKs are transferred into gnotobiotic mice and any alterations in fetal growth are measured. 3.3: To determine whether findings from gnotobiotic models can be applied to regulating fetal growth in a clinically relevant mouse model of FGR, previously identified bacterial taxa and biochemicals are applied to alleviate FGR in a protein-restriction mouse model.

### B1: Determining Whether the Maternal Microbiome Alters uNK Phenotype and Function

FIG. 8 shows that the angiogenic population of uNKs is reduced during mid-gestation in microbiota-depleted mice (FIG. 8).

Two models are used to study microbiota depletion: i) mice are maintained GF in isolators for life; ii) SPF mice are administered broad-spectrum antibiotics (ABX) twice daily for one week (metronidazole, ampicillin, neomycin and vancomycin), and maintained on antibiotic-supplemented drinking water (ampicillin, neomycin and vancomycin) for the duration of experimentation. Confocal imaging (1.1; 1.3): Murine uNKs are immunoreactive against Dolichos biflorus Agglutinin (DBA)-lectin, and categorized as angiogenic (DBA⁺) or IFNg-producing, non-angiogenic (DBA⁻)⁽⁴⁷⁻⁴⁹⁾. To characterize uNK phenotype (1.1) and effector function (1.3), gd10.5 and gd14.5 placenta sections are stained against DBA-lectin, Eomesodermin (Eomes; NK-specific transcription factor) and VEGFA, and scanned by confocal microscopy. UNK cell numbers, spatial localization, morphology, decidual density and VEGFA-colocalization are then quantified using ImageJ software. Flow cytometry (1.1; 1.3): UNKs are characterized by a defined receptor repertoire by flow cytometry^{(5.1)}. Placentae from gd10.5 and gd14.5 are mechanically dissociated to a single-cell suspension, and the leukocyte population enriched by density-gradient centrifugation. To characterize uNK phenotype (1.1), cluster of differentiation-(CD)45⁺CD3⁻CD122⁺ cells are stained against CD335 (NKp46), CD11b, CD27, CD49b, NK1.1, killer cell lectin-like receptor G1 (KLRG1), Ly49D and DBA. Relative abundance and absolute cell count are then calculated. UNK effector function (1.3) is induced by stimulation with phorbol 12-myristate 13-acetate (PMA) and Ionomycin combined with BrefeldinA treatment to block protein trafficking from the golgi apparatus. Following stimulation, expression of Interleukin (IL)-22, granulocyte-macrophage colony-stimulating factor (GM-CSF), tumor necrosis factor-α (TNFα), and IFNγ are measured. All analyses are performed using FlowJo software. Single-cell RNA sequencing (scRNASeq; 1.2): Placentae are collected from gd10.5 and gd14.5 SPF, GF and ABX mice and dissociated to single-cell suspensions, working from established methods⁽⁵²⁻⁵⁴⁾. These suspensions are then subjected to Illumina sequencing using 10× Genomics single-cell technology. Bioinformatics analysis is then performed using CellRanger software to distinguish cell types based on transcriptional signature. Metabolite in vivo supplementation (1.3): FIG. 13 and FIG. 14 show that primary and secondary bile salts, along with daidzein and genistein glucuronides, are reduced in GF and ABX mice relative to SPF controls. To evaluate the immunoregulatory capacity of microbiome-dependent metabolites, SPF and ABX mice are supplemented with either i) bile salts, or ii) assorted candidate chemical species identified in our metabolomics analysis, throughout gestation. UNK phenotype and effector function are assessed using confocal microscopy and flow cytometry as described above. Metabolite in vitro supplementation (1.3): As in the in vivo system, metabolite-dependent immunoregulation is assessed using cultured uNKs. Briefly, uNKs are isolated from placental single-cell suspensions after negative-selection against CD3ε and positive-selection against NKp46. Previously described metabolites are then supplemented into culture media, and supernatant concentration of cytokines (IFNγ, Interleukin (IL)-1β, TNFα, IL-22 and GM-CSF) and growth factors (VEGFA, P1GF, osteopontin, osteoglycin, and pleiotrophin⁽²⁸⁾) are quantified by ELISA.

Flow cytometric and histological analyses show that microbiome-depleted gd14.5 placentae have lower frequency of DBA-lectin⁺ uNKs compared to SPF littermates (FIG. 8D-F), Interestingly, total numbers of Eomes⁺ uNKs in gd14.5 GF and ABX placentae are also reduced relative to SPF littermates by histological analysis (FIG. 8G-H). These data suggest that the maternal microbiome regulates uNK abundance in the mid-gestation decidua.

### B2: Examine Roles for the Maternal Microbiome in Regulating Placental Vascular Development

FIG. 11 shows that GF and ABX mice have impaired feto-placental vasculature. The focus of this study is to determine whether the maternal microbiome is a critical regulator of placental vascular development, and whether regulatory effects are induced directly by microbiome-dependent biochemicals or by dysfunctional uNKs in the microbiome-depleted decidua (FIG. 8).

(2.1): Placental vascular ECs have been characterized using the EC marker, CD31, and the basement-membrane protein, laminin⁽⁵⁸⁾. Feto-placental vascular structure is visualized using confocal imaging of CD31- and laminin-stained placental tissues from gd10.5 and gd14.5 SPF, GF and ABX mice. Fetal labyrinth vasculature is quantified as fluorescence intensity normalized to total labyrinth area using ImageJ software using three sections per sample. μCT of fetoplacental vasculature (2.1): Visualization of feto-placental arterial vasculature is achieved as previously described⁽⁵⁹⁻⁶¹⁾. Briefly, individual gd14.5 conceptuses are dissected and a glass micropipette inserted into the umbilical artery. Heparinized PBS with 2% lidocaine is then perfused to clear the feto-placental vasculature, followed by perfusing MICROFIL, a radio-opaque casting compound. Once MICROFIL solidifies, samples are scanned at 8 μm-resolution, and a 3-dimensional reconstruction created using Vaa3d software and analyzed for volume, surface area and branching morphology. μCT of maternal vasculature (2.1): To analyze changes in maternal vascular structure and remodeling, a glass micropipette is inserted into the descending aorta⁽⁶²⁾ of euthanized gd14.5 SPF, GF and ABX dams, and the maternal vasculature is perfused as described above. Following setting of the MICROFIL casting compound, intact utero-placental vasculature is scanned as previously described. Uterine-, radial- and spiral-arteries from 3-dimensional reconstructions are then analyzed for number, length, cross-sectional area and resistance (based on Poiseuille's law⁽⁵⁹⁾). Metabolite in vivo supplementation (2.2): Microbiome-dependent metabolites described in A1 are administered to SPF and ABX mice beginning on the gd0.5 (observation of copulation plug), and continued through gestation. To assess the angiogenic potential of these metabolites, gd10.5 and gd14.5 CD31- and laminin-stained placentae are analyzed by confocal microscopy as described above. Metabolite in vitro supplementation (2.2): To evaluate novel angiogenic potential of previously described microbiome-dependent metabolites, primary ECs are isolated from SPF, GF and ABX placentae. Briefly, ECs are isolated from mechanically dissociated SPF, GF and ABX placentae following enzymatic digestion. Primary ECs are then cultured using standard conditions (In vitro angiogenesis assay kit, Abcam #ab204726), and supplemented with VEGF (positive control), Vinblastine (inhibitor-control) and microbiome-dependent metabolites. Cultured primary cells are then fixed, stained against CD31, scanned by confocal microscopy and analyzed for proliferation and morphology using ImageJ software. Adoptive cell transfer: To assess whether microbiota-depletion results in uNK-intrinsic dysfunction, adoptive transfer of SPF splenocytes⁽⁶³⁾ is performed into ABX recipients, as well as the reciprocal transfer, and assess maternal and fetal vasculature as described above. Briefly, recipient dams are injected with anti-asialo GM-1 on gd0.5 and gd3.5 to reduce the endogenous NK population. Donor splenocytes, stained with 5-chloromethylfluorescein diacetate (CMFDE), are injected into recipient dams on gd6.0, when peripheral NK cells are actively recruited to the uterine and decidual tissues^{(46, 47)}. To ensure successful transfer and recruitment of NKs to the uterus and decidua, CMFDE⁺ positive decidual cells are analyzed for co-localization with Eomes⁺ nuclei. Maternal and fetal vasculature are analyzed as described above.

To determine whether the maternal microbiome shapes feto-placental vascular development and structure, gd14.5 SPF, GF and ABX tissues were stained against CD31 (FIG. 11F). Quantification of CD31⁺ fetal labyrinth microvasculature reveals that maternal microbiota depletion reduces EC staining relative to total labyrinth area (FIG. 11G). This reduction in CD31⁺ ECs is coincident with qualitatively web-like vascular structure (FIG. 11F). To expand on the histological analysis, μCT was also performed to reconstruct 3-dimensional scans of feto-placental arterial vasculature at 8 μm resolution from SPF, GF and ABX conceptuses (FIG. 11H). GF and ABX feto-placental arterial volume (FIG. 11I) and surface area (FIG. 11J) is reduced relative to SPF controls, suggesting that the maternal microbiome is a critical factor for placental vascular development.

### B3: Assessing Mechanistic Influences of the Maternal Microbiome on Fetal Growth

Data shows that depleting the maternal microbiome alters both the maternal serum metabolome (FIG. 13, FIG. 14) and fetal serum metabolome (FIG. 13, FIG. 14), and that only transient depletion (ABX) of the microbiota results in FGR at mid-gestation (FIG. 3, FIG. 5).

(3.1): To evaluate whether microbiome-dependent metabolites described in A1 regulate placental and fetal growth, gd14.5 placentae and fetuses are weighed following previously described experiments in SPF and ABX mice. In addition to these groups of metabolites, a separate cohort of SPF and ABX pregnant dams are supplemented with metabolites identified in the fetal serum metabolome analysis, as these metabolites reflect fetal, rather than maternal, deficiencies. Select antibiotic treatment (3.1): Data show that fetal growth is reduced following broad-spectrum antibiotic treatment but not in GF mice (FIG. 3, FIG. 5). To assess whether this phenotype is due to off-target effects of absorbable antibiotics, mice are treated with either absorbable (Abs) or non-absorbable (NON) antibiotics. Mice are orally gavaged by either ampicillin and metronidazole (Abs), or neomycin and vancomycin (NON) twice daily for one week. Following initial depletion by oral gavage, Abs mice are maintained on ampicillin-supplemented drinking water, while NON mice are maintained on neomycin- and vancomycin-supplemented drinking water. 16S rDNA sequencing (3.1): To determine which bacterial taxa persist in the gut microbiota following select antibiotic treatment, fecal samples are collected from Abs and NON dams at gd0.5, gd10.5 and gd14.5 for 16S rDNA sequencing. Briefly, DNA are isolated from fecal samples using the MoBIO Powersoil kit, and the 16S rDNA V4-5 region is amplified by PCR using barcoded Caporaso primers⁽⁶⁶⁾. Samples are then sequenced by Illumina 2×250 bp paired-end sequencing, and analyzed with QIIME2 software to identify bacterial taxa. Gnotobiotic colonization (3.1): To determine whether bacterial taxa identified following select antibiotic treatment are sufficient to induce (NON) or prevent (Abs) FGR, type strains of candidate bacteria identified from 16S rDNA sequencing is obtained by the American Type Culture Collection (ATCC), the German Collection of Microorganisms and Cell Cultures (DSMZ) or RIKEN culture collections and grown under anaerobic conditions. Prior to mating, ABX mice are orally gavaged with 10⁹ colony forming units (CFU), and are either monocolonized or colonized with a combination of candidate bacteria.

Colonization of ABX gnotobiotic mice is confirmed by CFU plating and 16S rDNA sequencing. Gnotobiotic mice are then paired for breeding, and placental and fetal weights are measured as previously described. Adoptive transfer (3.2): In addition to secreting angiogenic growth factors, uNKs also secrete growth factors essential for normal fetal growth, including osteopontin, osteoglycin and pleiotrophin⁽²⁸⁾. Therefore, to test if microbiome-dependent uNK dysfunction contributes to reduced fetal growth in ABX mice, adoptive transfer experiments are performed as described above, and obtain gd14.5 fetal weights isolated from recipient dams. To assess the potential contributions from uNK-derived growth factors, osteopontin, osteoglycin and pleiotrophin transcripts are quantified (qPCR) and protein concentration is quantified (ELISA) from corresponding placentae. Protein-restriction model (3.3): To test whether the microbiome contributes to FGR in a protein-restriction (PR) diet, the microbiota compositions of mice on a PR diet are determined and compared to those on normal chow using 16S rDNA sequencing described above. Candidate bacteria are then purchased and grown as previously described, orally gavaged into ABX mice, and then maintained on normal chow. Fetal weights are then recorded to assess if the PR-associated microbiota contributes to FGR while dams are fed normal chow.

To address whether the maternal microbiome regulates bioavailability of circulating fetal metabolites, the fetal serum metabolome was analyzed (FIG. 13, FIG. 14). Compared to the maternal serum metabolome (FIG. 13, FIG. 14), fewer metabolites were dysregulated in GF and ABX fetuses, relative to SPF littermates (FIG. 13B), despite distinct clustering of microbiota-depleted groups by principle component analysis (FIG. 13D). Notably, metabolites from core metabolic pathways were comparable across SPF, GF and ABX fetal sera, suggesting no deviations in primary fetal metabolism or placental transport. The top 30 metabolites discriminating GF and ABX samples from SPF by random forest analysis were noted, with a predictive accuracy of 89-percent (FIG. 13J), and FIG. 14B shows overall subpathway changes in benzoate and histidine metabolism. In addition to metabolome changes, microbiota depletion coincides with reduced placental weight, relative to SPF and GF-CONV controls (FIG. 3A). Reduced placental weight was only accompanied by FGR in ABX mice (FIG. 3B), suggesting that mice born and reared GF are able to compensate for the observed placental insufficiencies. To verify whether these observations were due to off-target effects from absorbable antibiotics, mice were treated with either absorbable- or non-absorbable antibiotics as described above. Strikingly, only non-absorbable antibiotic treatment resulted in reduced placental and fetal weight (FIG. 5A-C), suggesting that FGR in this model is microbiome-dependent.

### REFERENCES FOR EXAMPLE 2

1. E. Maltepe, S. J. Fisher, Placenta: the forgotten organ. Annu Rev Cell Dev Biol 31, 523-552 (2015).
2. G. J. Burton, A. L. Fowden, Review: The placenta and developmental programming: balancing fetal nutrient demands with maternal resource allocation. Placenta 33 Suppl, S23-27 (2012).
3. M. R. Dilworth, C. P. Sibley, Review: Transport across the placenta of mice and women. Placenta 34 Suppl, S34-39 (2013).
4. G. Mor, I. Cardenas, The immune system in pregnancy: a unique complexity. Am J Reprod Immunol 63, 425-433 (2010).
5. G. Mor, I. Cardenas, V. Abrahams, S. Guller, Inflammation and pregnancy: the role of the immune system at the implantation site. Ann N Y Acad Sci 1221, 80-87 (2011).
6. C. W. Redman, Immunology of the placenta. Clin Obstet Gynaecol 13, 469-499 (1986).
7. M. PrabhuDas et al., Immune mechanisms at the maternal-fetal interface: perspectives and challenges. Nat Immunol 16, 328-334 (2015).
8. F. Staud, R. Karahoda, Trophoblast: The central unit of fetal growth, protection and programming. Int Biochem Cell Biol 105, 35-40 (2018).
9. K. L. Connor et al., Maternal metabolic, immune, and microbial systems in late pregnancy vary with malnutrition in mice. Biol Reprod, (2018).
10. M. Tomi, Y. Zhao, S. Thamotharan, B. C. Shin, S. U. Devaskar, Early life nutrient restriction impairs blood-brain metabolic profile and neurobehavior predisposing to Alzheimer's disease with aging. Brain Res 1495, 61-75 (2013).
11. L. E. Capron, P. G. Ramchandani, V. Glover, Maternal prenatal stress and placental gene expression of NR3C1 and HSD11B2: The effects of maternal ethnicity. Psychoneuroendocrinology 87, 166-172 (2018).
12. C. P. Gheorghe, R. Goyal, A. Mittal, L. D. Longo, Gene expression in the placenta: maternal stress and epigenetic responses. Int Dev Biol 54, 507-523 (2010).
13. C. P. Gheorghe, S. Mohan, K. C. Oberg, L. D. Longo, Gene expression patterns in the hypoxic murine placenta: a role in epigenesis? Reprod Sci 14, 223-233 (2007).
14. E. Y. Hsiao, P. H. Patterson, Activation of the maternal immune system induces endocrine changes in the placenta via IL-6. Brain Behav Immun 25, 604-615 (2011).
15. W. L. Wu, E. Y. Hsiao, Z. Yan, S. K. Mazmanian, P. H. Patterson, The placental interleukin-6 signaling controls fetal brain development and behavior. Brain Behav Immun 62, 11-23 (2017).
16. S. J. Fisher, Why is placentation abnormal in preeclampsia? Am J Obstet Gynecol 213, S115-122 (2015).
17. P. Hsu et al., Altered decidual DC-SIGN+ antigen-presenting cells and impaired regulatory T-cell induction in preeclampsia. Am J Pathol 181, 2149-2160 (2012).
18. I. L. Sargent, A. M. Borzychowski, C. W. Redman, NK cells and pre-eclampsia. J Reprod Immunol 76, 40-44 (2007).
19. A. Erlebacher, Immunology of the maternal-fetal interface. Annu Rev Immunol 31, 387-411 (2013).
20. V. Plaks et al., Matrix metalloproteinase-9 deficiency phenocopies features of preeclampsia and intrauterine growth restriction. Proc Natl Acad Sci USA 110, 11109-11114 (2013).
21. A. Robson et al., Uterine natural killer cells initiate spiral artery remodeling in human pregnancy. FASEB J 26, 4876-4885 (2012).
22. J. Hanna et al., Decidual NK cells regulate key developmental processes at the human fetal-maternal interface. Nat Med 12, 1065-1074 (2006).
23. M. J. Guimond, B. Wang, B. A. Croy, Engraftment of bone marrow from severe combined immunodeficient (SCID) mice reverses the reproductive deficits in natural killer cell-deficient tg epsilon 26 mice. J Exp Med 187, 217-223 (1998).
24. B. A. Croy et al., Imaging of vascular development in early mouse decidua and its association with leukocytes and trophoblasts. Biol Reprod 87, 125 (2012).
25. C. Tayade et al., Genetic deletion of placenta growth factor in mice alters uterine NK cells. J Immunol 178, 4267-4275 (2007).
26. P. D. Lima, J. Zhang, C. Dunk, S. J. Lye, B. A. Croy, Leukocyte driven-decidual angiogenesis in early pregnancy. Cell Mot Immunol 11, 522-537 (2014).
27. G. E. Lash et al., Expression of angiogenic growth factors by uterine natural killer cells during early pregnancy. J Leukoc Biol 80, 572-580 (2006).
28. B. Fu et al., Natural Killer Cells Promote Fetal Development through the Secretion of Growth-Promoting Factors. Immunity 47, 1100-1113 e1106 (2017).
29. T. S. Stappenbeck, L. V. Hooper, J. I. Gordon, Developmental regulation of intestinal angiogenesis by indigenous microbes via Paneth cells. Proc Natl Acad Sci USA 99, 15451-15455 (2002).
30. Z. Jie et al., The gut microbiome in atherosclerotic cardiovascular disease. Nat Commun 8, 845 (2017).
31. W. H. Tang, S. L. Hazen, The Gut Microbiome and Its Role in Cardiovascular Diseases. Circulation 135, 1008-1010 (2017).
32. V. Braniste et al., The gut microbiota influences blood-brain barrier permeability in mice. Sci Transl Med 6, 263ra158 (2014).
33. A. Sehgal, P. Murthi, J. E. Dahlstrom, Vascular changes in fetal growth restriction: clinical relevance and future therapeutics. J Perinatol, (2018).
34. B. N. Radford, V. K. M. Han, Offspring from maternal nutrient restriction in mice show variations in adult glucose metabolism similar to human fetal growth restriction. J Dev Orig Health Dis, 1-10 (2018).
35. Y. Belkaid, O. J. Harrison, Homeostatic Immunity and the Microbiota. Immunity 46, 562-576 (2017).
36. T. C. Fung, C. A. Olson, E. Y. Hsiao, Interactions between the microbiota, immune and nervous systems in health and disease. Nat Neurosci 20, 145-155 (2017).
37. A. J. Macpherson, M. G. de Aguero, S. C. Ganal-Vonarburg, How nutrition and the maternal microbiota shape the neonatal immune system. Nat Rev Immunol 17, 508-517 (2017).
38. C. A. Thaiss, N. Zmora, M. Levy, E. Elinav, The microbiome and innate immunity. Nature 535, 65-74 (2016).
39. N. Arpaia et al., Metabolites produced by commensal bacteria promote peripheral regulatory T-cell generation. Nature 504, 451-455 (2013).
40. D. Erny et al., Host microbiota constantly control maturation and function of microglia in the CNS. Nat Neurosci 18, 965-977 (2015).
41. G. Storelli et al., Lactobacillus plantarum promotes Drosophila systemic growth by modulating hormonal signals through TOR-dependent nutrient sensing. Cell Metab 14, 403-414 (2011).
42. M. Schwarzer et al., Lactobacillus plantarum strain maintains growth of infant mice during chronic undernutrition. Science 351, 854-857 (2016).
43. M. I. Smith et al., Gut microbiomes of Malawian twin pairs discordant for kwashiorkor. Science 339, 548-554 (2013).
44. F. Drissi, D. Raoult, V. Merhej, Metabolic role of lactobacilli in weight modification in humans and animals. Microb Pathog 106, 182-194 (2017).
45. B. A. Croy et al., Uterine natural killer cells: insights into their cellular and molecular biology from mouse modelling. Reproduction 126, 149-160 (2003).
46. L. M. Gaynor, F. Colucci, Uterine Natural Killer Cells: Functional Distinctions and Influence on Pregnancy in Humans and Mice. Front Immunol 8, 467 (2017).
47. Z. Chen et al., DBA-lectin reactivity defines mouse uterine natural killer cell subsets with biased gene expression. Biol Reprod 87, 81 (2012).
48. A. A. Ashkar, J. P. Di Santo, B. A. Croy, Interferon gamma contributes to initiation of uterine vascular modification, decidual integrity, and uterine natural killer cell maturation during normal murine pregnancy. J Exp Med 192, 259-270 (2000).
49. C. Wang et al., Expression of vascular endothelial growth factor by granulated metrial gland cells in pregnant murine uteri. Cell Tissue Res 300, 285-293 (2000).
50. S. C. Ganal et al., Priming of natural killer cells by nonmucosal mononuclear phagocytes requires instructive signals from commensal microbiota. Immunity 37, 171-186 (2012).
51. H. Yadi et al., Unique receptor repertoire in mouse uterine NK cells. J Immunol 181, 6140-6147 (2008).
52. A. C. Nelson, A. W. Mould, E. K. Bikoff, E. J. Robertson, Single-cell RNA-seq reveals cell type-specific transcriptional signatures at the maternal-foetal interface during pregnancy. Nat Commun 7, 11414 (2016).
53. M. Pavlicev et al., Single-cell transcriptomics of the human placenta: inferring the cell communication network of the maternal-fetal interface. Genome Res 27, 349-361 (2017).
54. Y. Liu et al., Single-cell RNA-seq reveals the diversity of trophoblast subtypes and patterns of differentiation in the human placenta. Cell Res 28, 819-832 (2018).
55. S. Fiorucci, M. Biagioli, A. Zampella, E. Distrutti, Bile Acids Activated Receptors Regulate Innate Immunity. Front Immunol 9, 1853 (2018).
56. Y. Zhang, T. T. Song, J. E. Cunnick, P. A. Murphy, S. Hendrich, Daidzein and genistein glucuronides in vitro are weakly estrogenic and activate human natural killer cells at nutritionally relevant concentrations. J Nutr 129, 399-405 (1999).
57. Y. E. Wu, L. Pan, Y. Zuo, X. Li, W. Hong, Detecting Activated Cell Populations Using Single-Cell RNA-Seq. Neuron 96, 313-329 e316 (2017).
58. I. Tai-Nagara et al., Placental labyrinth formation in mice requires endothelial FLRT2/UNCSB signaling. Development 144, 2392-2401 (2017).
59. M. Y. Rennie, A. Rahman, K. J. Whiteley, J. G. Sled, S. L. Adamson, Site-specific increases in utero- and fetoplacental arterial vascular resistance in eNOS-deficient mice due to impaired arterial enlargement. Biol Reprod 92, 48 (2015).
60. M. Y. Rennie, K. J. Whiteley, S. Kulandavelu, S. L. Adamson, J. G. Sled, 3D visualisation and quantification by microcomputed tomography of late gestational changes in the arterial and venous feto-placental vasculature of the mouse. Placenta 28, 833-840 (2007).
61. A. C. Langheinrich et al., Quantitative 3D micro-CT imaging of the human feto-placental vasculature in intrauterine growth restriction. Placenta 29, 937-941 (2008).
62. M. Y. Rennie, K. J. Whiteley, S. L. Adamson, J. G. Sled, Quantification of Gestational Changes in the Uteroplacental Vascular Tree Reveals Vessel Specific Hemodynamic Roles During Pregnancy in Mice. Biol Reprod 95, 43 (2016).
63. B. A. Croy, X. Xie, In vivo models for studying homing and function of murine uterine natural killer cells. Methods Mol Med 122, 77-92 (2006).
64. T. E. O'Sullivan et al., Adipose-Resident Group 1 Innate Lymphoid Cells Promote Obesity-Associated Insulin Resistance. Immunity 45, 428-441 (2016).
65. K. K. Bhasin et al., Maternal low-protein diet or hypercholesterolemia reduces circulating essential amino acids and leads to intrauterine growth restriction. Diabetes 58, 559-566 (2009).
66. J. G. Caporaso et al., Ultra-high-throughput microbial community analysis on the Illumina HiSeq and MiSeq platforms. ISME J 6, 1621-1624 (2012).

### Example 3: Microbiome-Based Intervention for Intrauterine Growth Restriction

The following experimental results, presented through figures, illustrate the potential for live biotherapeutic or microbial metabolite treatment for intrauterine growth restriction, preeclampsia, or related obstetric issues.

The maternal microbiota regulates placental and fetal development as shown through the results in FIG. 3, which has the following panels: (A) Average E14.5 placental weights from SPF (n=10), GF (n=8), ABX (n=10) and GF-CONV (n=7) litters. (B) Average E14.5 fetal weights from litters represented in (A). (C) Fetal to placental weight ratios from litters used in (A-B). (D) Correlation analysis of litters represented in (A-B). (E) Average P0 neonatal weights of SPF (n=6), GF (n=6), ABX (n=5) and GF-CONV (n=2). (F) Representative images hematoxyling and eosin staining of E14.5 SPF, GF and ABX placentae. (G) Enlarged inset images from hematoxyling and eosin staining of E14.5 fetal labyrinth displayed in (F). (H) Representative images of E14.5 SPF, GF, ABX and GF-CONV fetuses reconstructed using micro-computed tomography (μCT) and quantification of total fetal volume (I), fetal brain volume (K), fetal brain ventricle volume (K), brain to body volume ratios (L), and fetal surface area (M).

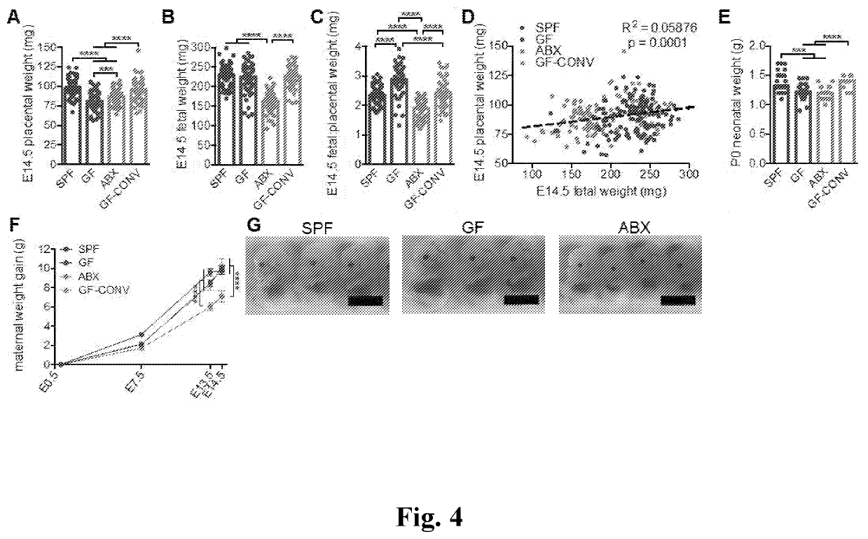

The maternal microbiota regulates placental and fetal development as shown through the results in FIG. 4, which has the following panels: (A-E) Analysis of individual placentae and fetuses from litters described in FIG. 3. (F) Maternal weight gain assessed at E0.5, E7.5, E13.5 and E14.5 in SPF (n=9), GF (n=2), ABX (n=7) and GF-CONV (n=6) dams. (G) Representative images of gross morphological features of E14.5 SPF, GF and ABX fetuses (scale=1 cm).

Reduced placental and fetal weight is not due to off-target effects of absorbable antibiotics as shown through the results in FIG. 5, which has the following panels: (A) Average placental weights from E14.5 SPF (same as FIG. 3A), ABX (n=9), non-absorbable antibiotic treated (Non-Abs; n=4) and absorbable-antibiotic treated (Abs; n=5) litters. (B) Average E14.5 fetal weights from litters represented in (A). (C) Average fetal to placental weight ratios from litters used in (A-B). (D) Correlation analysis of individual placentae and fetuses in litters represented in (A-B). (E-G) Analyses of individual placentae and fetuses from litter group analyses represented in (A-C). (H) Maternal weight gain assessed at E0.5, E7.5, E13.5 and E14.5 in ABX (n=9), Non-abs (n=4) and Abs (n=3) dams.

Candidate bacterial taxa associated with reduced placental and fetal growth deficits revealed by 16S sequencing as shown through the results in FIG. 6, which has the following panels: (A) Taxonomic identities of bacterial 16S ribosomal DNA sequencing isolated from E14.5 SPF (n=3), non-absorbable antibiotic treated (Non-Abs; n=5), and absorbable antibiotic treated (Abs; n=5) fecal samples. (B) Alpha diversity rarefaction plots from samples described in (A). (C) Beta diversity determined by weighted-Unifrac analysis of samples described in (A).

Depletion of the maternal microbiota alters uterine natural killer (uNK) abundance in the E14.5 decidua as shown through the results in FIG. 8, which has the following panels: (A) Representative images of E14.5 deciduae from SPF, GF and ABX mice stained against Dolichos Biflorus Aggultinin-lectin (DBA) and DAPI. (B) Representative images of E14.5 deciduae from SPF, GF and ABX mice stained against DBA and Eomesodermin (Eomes). (C) Quantification of images represented in (A-B), demonstrating numbers of cells stained positive for DBA only and for DBA co-localized with Eomes. (D) Representative frequencies of DBA expression in E14.5 SPF, GF and ABX deciduae, gated on live CD45+, CD3−, CD122+ cells. (E) Quantification of DBA positive and negative frequencies as shown in (D) from SPF (n=5), GF (n=5) and ABX (n=6) deciduae.

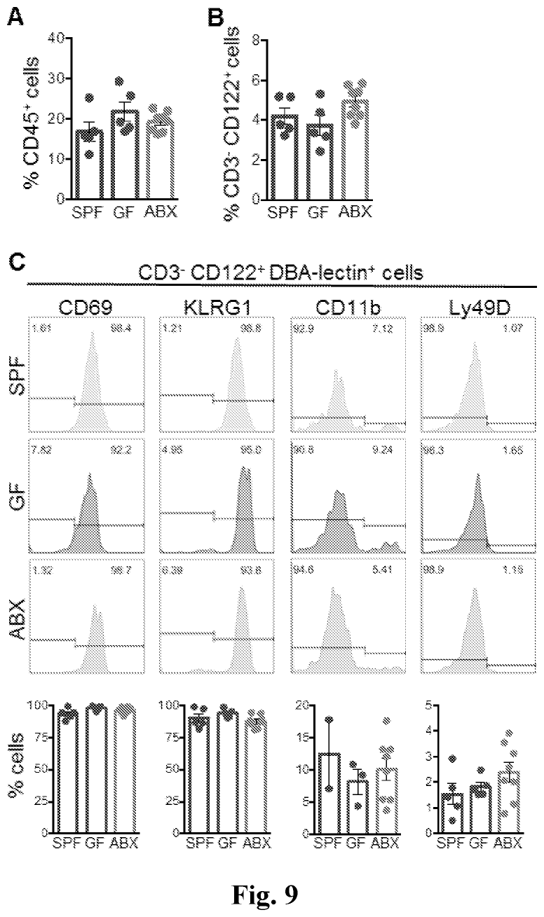

Maternal microbiota depletion does not alter uNK maturation phenotypes as shown through the results in FIG. 9, which has the following panels: (A) Frequency of CD45+ cells in E14.5 deciduae from SPF (n=5), GF (n=5) and ABX (n=8) mice, determined by flow cytometry. (B) Frequency of CD3− CD122+ cells in E14.5 deciduae from SPF (n=5), GF (n=5) and ABX (n=8) mice, determined by flow cytometry. (C) Expression of uNK markers CD69, KLRG1, CD11b and Ly49D from SPF (n=5), GF (n=5) and ABX (n=8) E14.5 deciduae.

The maternal microbiota promotes placental vascular development as shown through the results in FIG. 11, which has the following panels: (A) Representative feto-placental arterial vascular reconstructions by micro-computed tomography of E14.5 SPF, GF, ABX and GF-CONV placentae. Quantification of E14.5 feto-placental arterial vasculature from SPF (n=9), GF (n=9), ABX (n=6) and GF-CONV (n=5) volume (B) and surface area (C). (D) Histological analysis of feto-placental microvasculature in E14.5 SPF, GF and ABX placentae stained against laminin, TER119 and DAPI. (E) Quantification of raw integrated density of laminin staining in the fetal labyrinth in SPF (n=6), GF (n=7) and ABX (n=6) placentae.

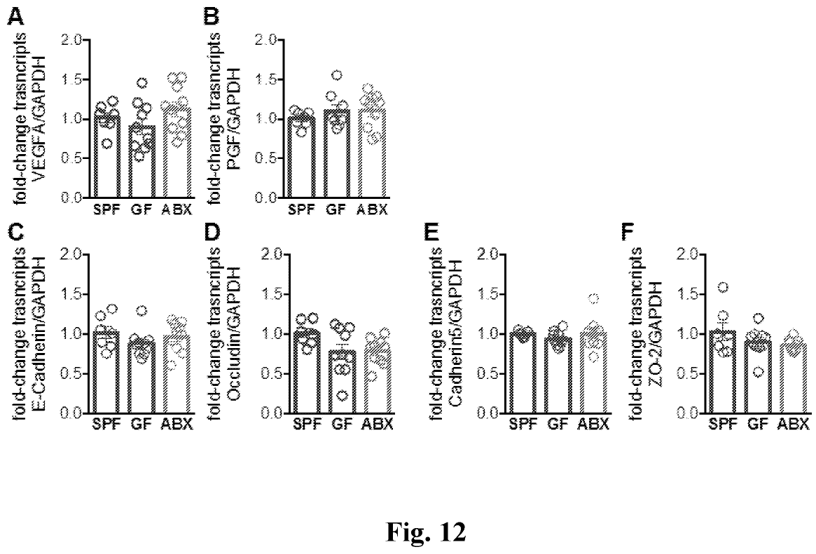

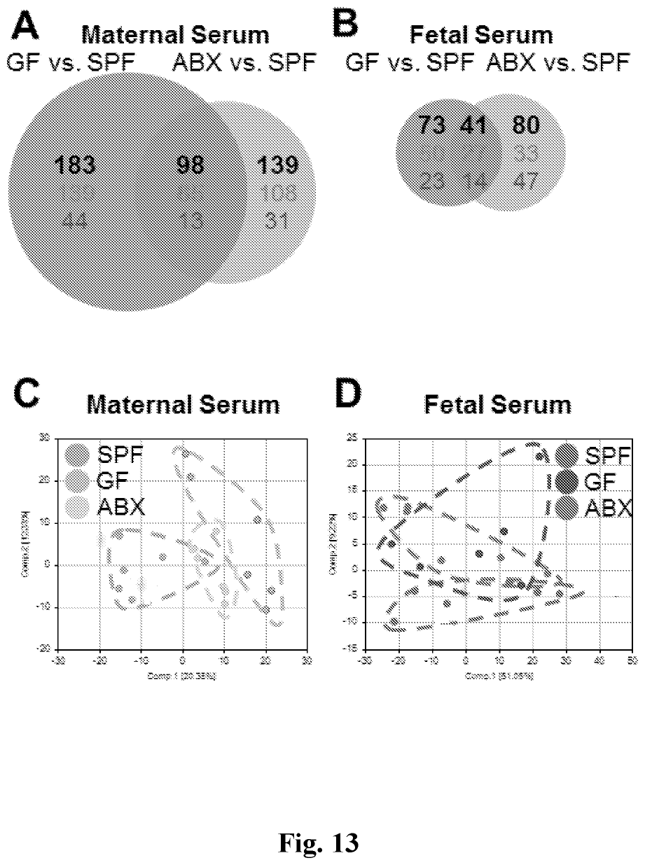

Microbiota depletion does not vascular growth factor or barrier-related transcript expression in the E14.5 placenta as shown through the results in FIG. 12, which has the following panels: Transcript expression in E14.5 SPF (n=7), GF (n=10) and ABX (n=10) placentae for VEGFA (A), PGF (B), ECadherin (C), Occludin (D), Cadherin-5 (E) and ZO-2 (F). (G) Histological analysis of ZO-1 immunoreactivity in E14.5 junctional zone of SPF, GF, ABX and GF-CONV E14.5 placentae, and quantification of ZO-1 fluorescence (H). (I) ZO1 transcript relative abundance in SPF, GF, ABX and GF-CONV E14.5 placentae.

The maternal microbiota modulates the maternal and fetal sera metabolomes as shown through the results in FIG. 13, which has the following panels: Venn diagrams demonstrating significantly altered metabolites (black text), both downregulated (red text) and upregulated (blue text) in SPF (n=6), GF (n=6) and ABX (n=6) maternal serum (A) and fetal serum (B). Principle component analyses of maternal serum (C) and fetal serum (D) shown in (A-B). Truncated global metabolite profile changes in GF maternal serum relative to SPF (E), and ABX maternal serum relative to SPF (F). Truncated global metabolite profile changes in GF fetal serum relative to SPF (G), and ABX fetal serum relative to SPF (H). Top thirty metabolites defined by random forest analysis of maternal serum (I) and fetal serum (J).

The maternal microbiome regulates metabolic subpathways in maternal and fetal sera as shown through the results in FIG. 14, which has the following panels: (A) Subpathway fold-enrichment analysis of GF and ABX maternal serum samples shown in FIG. 13A. (B) Subpathway fold-enrichment analysis of GF and ABX fetal serum samples shown in FIG. 13B.

### Example 4: Metabolite Supplementation Studies

### Microbiome-Dependent Metabolite Supplementation

To assess whether microbiome-dependent metabolites are sufficient to rescue placental immune and vascular homeostasis we observed in gnotobiotic mice, we supplemented broad-spectrum antibiotic-treated (ABX) mice with a mixture of metabolites we defined in our metabolomics analyses (see Table 1). Physiological concentrations for each metabolite were determined in mouse, rat or human literature, and multiplied by 24 to account for the single injection given in the 24-hour period. Briefly, ABX females were isolated upon observation of the copulation plug at embryonic day 0.5 (E0.5), and received once daily injections of the metabolite mixture reconstituted with sterile phosphate buffer saline (PBS), or PBS-only vehicle controls. Dams were sacrificed by cervical dislocation at E14.5, and tissues were collected and analyzed as previously described for further application.

### Short-Chain Fatty Acid (SCFA) Supplementation

To determine whether microbiota-produced SCFAs are sufficient to rescue placental immune and vascular homeostasis in microbiota-depleted mice, we supplemented SCFAs in drinking water and gave ad libitum access to isolated dams upon observation of the copulation plug at E0.5. For our SCFA mixture, we administered sodium propionate (25 mM), sodium butyrate (40 mM) and sodium acetate (67.5 mM) in sterile water as previously described (Erny et al, 2015; Smith et al, 2013). Dams were sacrificed by cervical dislocation at E14.5, and tissues were collected and analyzed as previously described for further application.

Table 1 provided below shows the metabolite concentrations used in the in vivo supplementation experiments.

TABLE 1

| | physiological | final |
|---|---|---|
| | concentration | concentration for |
| metabolite name | (nM) | pooling (uM) |
| imidazole propionate | 100 | 29.64 |
| N,N,N-trimethy1-5-aminovalerate | 1800 | 714.096 |
| 4-hydroxyphenylacetate | 50 | 13.452 |
| phenol sulfate | 700 | 316.008 |
| indolepropionate | 950 | 428.868 |
| indoxyl glucuronide | 1000 | 360.24 |
| N-methylproline | 1000 | 323.76 |
| phenylacetylglycine | 1500 | 595.08 |
| trimethylamine N-oxide | 2500 | 957.6 |
| taurodeoxycholate | 260 | 118.56 |
| biotin | 630 | 160.8768 |
| hippurate | 2000 | 893.76 |
| 2-(4-hydroxyphenyl)propionate | 100 | 42.864 |
| cinnamoylglycine | 487 | 222.072 |
| equol glucuronide | 0.6 | 0.2736 |
| equol sulfate | 0.6 | 0.2736 |
| 2-aminophenol sulfate | 1000 | 433.2 |
| 3-indoxyl sulfate | 6000 | 1559.52 |
| p-cresol sulfate | 600 | 268.128 |

### Example 5: Bacterial Taxa and Bacterial SCFA Associated with Placental and Fetal Growth

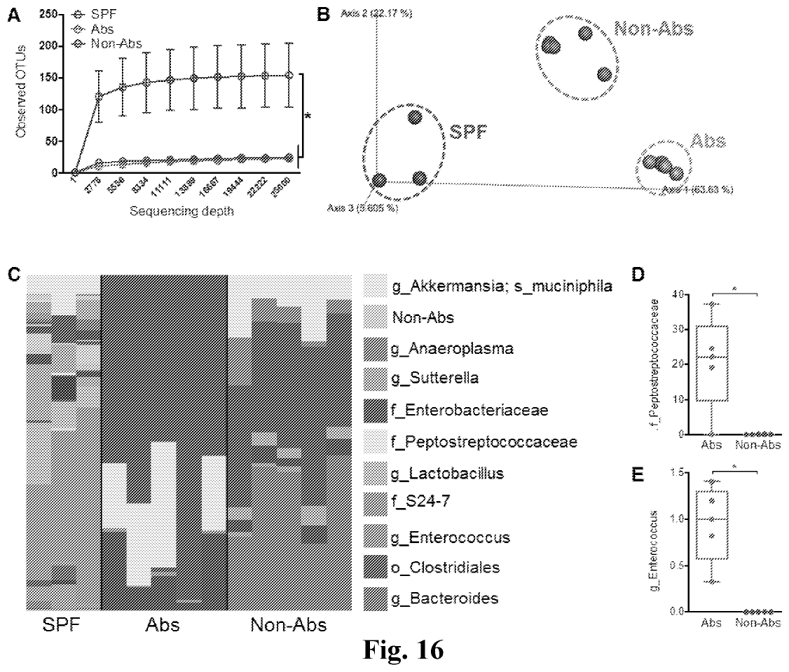

Candidate bacterial taxa associated with reduced placental and fetal growth deficits revealed by 16S sequencing as shown through the results in FIG. 16, which has the following panels: (A) Alpha rarefaction plots demonstrating alpha-diversity based on observed OTUs from 16S sequencing of SPF, Abs and Non-Abs E14.5 fecal microbiota. (B) Weighted UniFrac analysis demonstrating beta-diversity of SPF (n=3), Abs (n=5) and Non-Abs (n=5) E14.5 fecal microbiota communities represented in (A). (C) Taxa bar plots demonstrating relative sequence abundance of bacterial taxa from SPF, Abs and Non-Abs E14.5 fecal microbiota communities represented in (A). (D) Relative abundance of family Peptostreptococcaceae in Abs and Non-Abs E14.5 fecal microbiota communities represented in (A). (E) Relative abundance of genus Enterococcus in Abs and Non-Abs E14.5 fecal microbiota communities represented in (A).

Bacterial taxa or particular genera/species thereof associated with reduced placental and fetal growth deficits as revealed in these experiments can be used in various embodiments disclosed herein to treat a variety of conditions as disclosed herein, for example by administering such bacterial taxa or particular genera/species thereof to a subject having a condition or to a subject to prevent a condition in the subject or in an unborn baby of the subject.

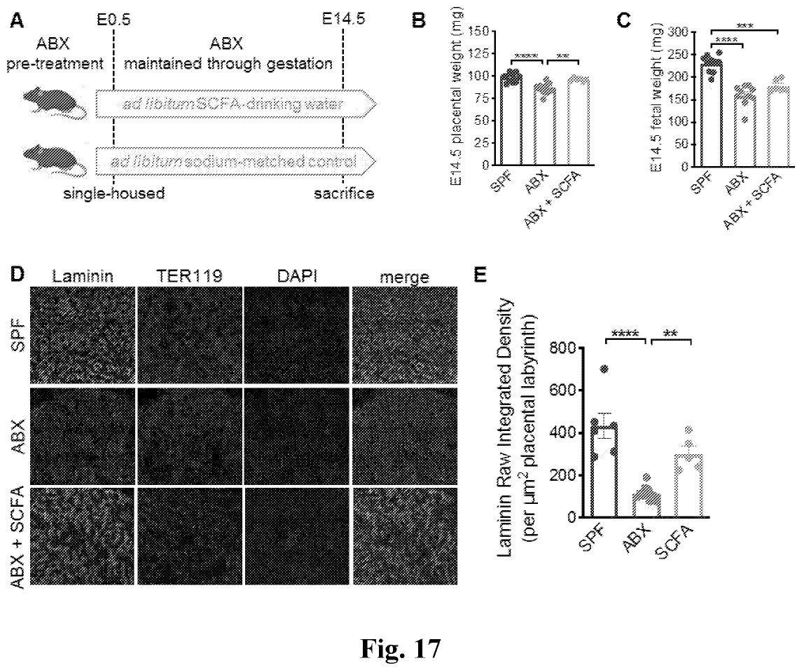

Bacterial SCFA supplementation restores placental weight and microvascular impairments following maternal microbiota depletion as shown through the results in FIG. 17, which has the following panels: A) Schematic representation of SCFA administration to ABX dams. B) E14.5 placental weight by litter average for SPF (same as FIG. 1A), ABX (same as FIG. 1A) and ABX+SCFA (n=6). C) E14.5 fetal weight by litter average for litters shown in FIG. 6B. D) Representative images of microvasculature staining panel against laminin, TER119 and DAPI. E) Quantification of fetal labyrinth laminin raw integrated density normalized to fetal labyrinth area for litters shown in FIG. 6A-6B.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method comprising administering to a subject a composition comprising:
a compound selected from primary bile acids, secondary bile acids, daidzein, genistein, osteopontin, osteoglycin, pleiotrophin, tyrosine, histidine, peptides, benzoate, tryptophan, 2-aminophenol sulfate, catechol sulfate, 4-hydroxyphenylacetate sulfate, N,N,N-trimethyl-5-aminovalerate, phenol sulfate, equol sulfate, hippurate, p-cresol sulfate, imidazolepropionate, indolepropionate, equol glucuronide, valerylglycine, phenol glucuronide, salicylate, S-carboxymethyl-L-cysteine, and trimethylamine N-oxide, and metabolites and derivatives thereof, or a combination thereof; and/or
one or more bacterial species found in a maternal microbiome, selected from phylum Firmicutes, phylum Bacteroidetes, phylum Proteobacteria, or a combination thereof.

2. The method of claim 1, wherein
the method promotes healthy growth in an unborn baby and the subject is a maternal subject gestating the unborn baby;
the method promotes healthy growth in an unborn baby and the female subject is a fertile, ovulating female subject or a female subject seeking to implant an embryo;
the method promotes placental development in the subject who is a maternal subject gestating an unborn baby;
the method promotes placental development in a maternal subject gestating an unborn baby and the subject is a female subject;
the method promotes angiogenesis in a fetal subject and the subject is a maternal subject gestating the unborn baby;
the method promotes angiogenesis in a fetal subject and the subject is a fertile, ovulating female subject or a female subject seeking to implant an embryo;
the method inhibits development of preeclampsia or fetal growth restriction (FGR) in a maternal subject gestating an unborn baby and the subject is the maternal subject; or
the method inhibits development of preeclampsia or fetal growth restriction (FGR) in a maternal subject gestating an unborn baby, and the subject is a fertile, ovulating female subject or a female subject seeking to implant an embryo.

3. The method of claim 2, wherein promoting healthy growth comprises promoting an increase in one or more of fetal weight, fetal volume, fetal surface area, fetal brain volume, fetal ventricle volume, and fetal brain:body ratio.

4-5. (canceled)

6. The method of claim 2, wherein promoting placental development comprises promoting an increase in one or more of a placental angiogenesis, placental immunity, and placental weight.

7-8. (canceled)

9. The method of claim 2, wherein promoting angiogenesis in the fetal subject comprises promoting development of uterine natural killer (uNK) cells.

10-11. (canceled)

12. The method of claim 2, wherein the maternal subject or the female subject is characterized by a depleted maternal microbiome.

13. The method of claim 12, wherein the maternal subject or the female subject is germ-free (GF) or antibiotic-treated (ABX) prior to administration of the composition.

14. The method of claim 2, wherein the maternal subject or the female subject is a mammal.

15. The method of claim 14, wherein the mammal is a human.

16. The method of claim 15, wherein the composition is administered during the first trimester of gestation.

17. The method of claim 15, wherein the composition is administered during the second trimester of gestation.

18. The method of claim 15, wherein the composition is administered during the third trimester of gestation.

19. The method of claim 15, further comprising administering the composition to the maternal subject prior to gestation.

20. (canceled)

21. The method of claim 2, wherein the compound is selected from primary and secondary bile acids.

22. The method of claim 2, wherein the compound is selected from daidzein and genistein, and derivatives thereof.

23. The method of claim 2, wherein the compound is selected from osteopontin, osteoglycin, and pleiotrophin.

24. The method of claim 2, wherein the one or more bacteria in the composition are selected from phylum Firmicutes, phylum Bacteroidetes, phylum Proteobacteria, or a combination thereof.

25. The method of claim 24, wherein the one or more bacteria in the composition from phylum Firmicutes comprises one or more bacteria selected from class Clostridia.

26. The method of claim 24, wherein the one or more bacteria in the composition from phylum Bacteroidetes comprises one or more bacteria selected from genus Bacteroides (e.g., B. thetaiotaomicron, B. uniformis, B. vulgatus, B. ovatus, B. fragilus).

27. The method of claim 24, wherein the one or more bacteria in the composition from phylum Proteobacteria comprise one or more bacteria selected from family Enterobacteriaecea
